(19) United States
(12) Patent Application Publication
(10) Pub. No.: US 2013/0143210 A1
(43) Pub. Date: Jun. 6, 2013

(54) METHOD FOR THE PROGNOSIS OF OVARIAN CARCINOMA

(71) Applicants: Fabian Model, Penzberg (DE); Tamas Rujan, Lorrach (DE); Tobias Mayr, Berlin (DE); Andre Rosenthal, Ludwigsfelde (DE)

(72) Inventors: Fabian Model, Penzberg (DE); Tamas Rujan, Lorrach (DE); Tobias Mayr, Berlin (DE); Andre Rosenthal, Ludwigsfelde (DE)

(21) Appl. No.: 13/503,798

(22) PCT Filed: Oct. 28, 2010
(86) PCT No.: PCT/EP2010/066413

§ 371 Date: Apr. 26, 2012

(30) Foreign Application Priority Data
Oct. 28, 2009 (EP) 09075479.7

(51) Int. Cl. C12Q 1/68

(52) U.S. Cl. CPC C12Q 1/68
USPC 435/6.11; 536/23.1

(57) ABSTRACT

The invention relates to a method for determining prognosis of subjects with ovarian carcinoma using a biological sample comprising genomic tumor DNA isolated from the subject. According to the invention, the method comprises the steps of: determining the methylation status of a CpG dinucleotide in a target sequence that is selected from the group consisting of the target sequences as referred to by name in Table 1 in a biological sample isolated from a subject; and deducing from the determined methylation status of the target sequence the prognosis of subject with ovarian carcinoma. The improved prognosis determination of the subject with ovarian carcinoma enables the improved treatment of the said patient.

The present invention relates to genomic DNA sequences wherein CpG methylation patterns correlate with time to progression of a disease status. Particular embodiments provide methods, nucleic acids and kits useful for predicting the prognosis of ovarian carcinomas.

### BACKGROUND

DNA Methylation.

The etiology of pathogenic states is known to involve modified methylation patterns of individual genes or of the genome. 5-methylcytosine, in the context of CpG dinucleotide sequences, is the most frequent covalently modified base in the DNA of eukaryotic cells, and plays a role in the regulation of transcription, genetic imprinting, and tumorigenesis. The identification and quantification of 5-methylcytosine sites in a specific specimen, or between or among a plurality of specimens, is thus of considerable interest, not only in research, but particularly for the molecular diagnoses of various diseases.

Correlation of Aberrant DNA Methylation with Cancer.

Aberrant DNA methylation within CpG “islands” is characterized by hyper- or hypomethylation of CpG dinucleotide sequences leading to abrogation or overexpression of a broad spectrum of genes, and is among the earliest and most common alterations found in, and correlated with human malignancies. Additionally, abnormal methylation has been shown to occur in CpG-rich regulatory elements in intronic and coding parts of genes for certain tumors. In ovarian carcinoma, for example, aberrant DNA methylation constitutes one of the most prominent alterations and inactivates tumor suppressor genes such as ARH1, RASSF1A and DNA mismatch repair genes such as BRCA1 (Hennessy B T et al. Ovarian cancer: homeobox genes, autocrine/paracrine growth, and kinase signaling. Int J Biochem Cell Biol. 2006; 38(9):1450-6).

In contrast to the specific hypermethylation of tumor suppressor genes, an overall hypomethylation of DNA can be observed in tumor cells. This decrease in global methylation can be detected early, far before the development of a recognizable tumor. A correlation between hypomethylation and increased gene expression has been determined for many oncogenes.

Ovarian Carcinoma.

Ovarian cancer is the fifth most common cancer in women and the leading cause of death from gynecological cancers. It is estimated that approximately 21,650 new cases will be diagnosed 2008 in the US, with 15,520 of the women dying from this disease, corresponding to 6% of all cancer deaths. Worldwide there are more than 200,000 new cases of ovarian carcinoma each year. Studies in Europe and the US show that the average five-year survival rate is between 35% and 45%. As for most cancers, incidence is highest for women older than 60. Younger women are affected more often when a predisposition for the disease is inherited, such as by mutations in the BRCA1/2 or HPNCC genes. By far the most common cancers of the ovaries constitute adenocarcinomas of epithelial origin with serous, clear cell, endometrioid or mucous histological subtypes.

Treatment of most epithelial ovarian carcinomas consists of surgical removal of as much cancerous tissue as possible (debulking), both ovaries (bilateral salpingo oophorectomy) and the uterus (hysterectomy), followed by a first line combination chemotherapy with platinum typically carboplatin (sometimes cisplatin) and another agent, typically a taxane like paclitaxel.

Prognosis and Treatment of Ovarian Cancer.

Survival of ovarian cancer patients is influenced by several factors. The most significant independent prognostic factor is the stage of the disease when initially diagnosed. Ovarian cancer staging is by the FIGO staging system and uses information obtained after surgery, which can include a total abdominal hysterectomy, removal of (usually) both ovaries and fallopian tubes, (usually) the omentum, and pelvic (peritoneal) washings for cytopathology. The AJCC stage is the same as the FIGO stage. The AJCC staging system describes the extent of the primary Tumor (T), the absence or presence of metastasis to nearby lymph Nodes (N), and the absence or presence of distant Metastasis (M).

### Stage I—Limited to One or Both Ovaries

IA—involves one ovary; capsule intact; no tumor on ovarian surface; no malignant cells in ascites or peritoneal washings
IB—involves both ovaries; capsule intact; no tumor on ovarian surface; negative washings
IC—tumor limited to ovaries with any of the following: capsule ruptured, tumor on ovarian surface, positive washings

### Stage II—Pelvic Extension or Implants

IIA—extension or implants onto uterus or fallopian tube; negative washings
IIB—extension or implants onto other pelvic structures; negative washings
IIC—pelvic extension or implants with positive peritoneal washings
   Stage III—Microscopic Peritoneal Implants Outside of the Pelvis; or Limited to the Pelvis with Extension to the Small Bowel or Omentum
IIIA—microscopic peritoneal metastases beyond pelvis
IIIB—macroscopic peritoneal metastases beyond pelvis less than 2 cm in size
IIIC—peritoneal metastases beyond pelvis >2 cm or lymph node metastases

### Stage IV—Distant Metastases to the Liver or Outside the Peritoneal Cavity

More than 90% survive longer than 5 years, when the disease is diagnosed while still confined to the ovaries. With regional metastases, the five-year survival is at 70%, while disease that is detected with distant metastasis shows a 25% 5-year-survival rate. A further independent and important prognostic factor is the success of the debulking surgery (i.e., the amount of residual disease)—a factor that cannot be altered by analytical approaches. Another important prognostic factor is the histopathological subtype. There are several histological subtypes including serous, endrometroid, mucinous, clear cell and papillary. However, most of the ovarian epithelial carcinomas are of the serous subtype.

The most significant secondary prognostic factor is the response rate to the first line platinum treatment.

The treatment of ovarian cancer patients today is mostly done without taking prognostic or predictive factors into account. The only exception is stage 1 ovarian cancer (15% of the ovarian cancer cases) where stage and grade of the disease, the histopathologic type, and the patient's age and overall health are used for treatment decision.

In all events, after surgical cytoreduction of the ovaries, standard first line treatment consists of a platinum and taxane combination that shows the best clinical survival data.

Unfortunately, ovarian cancer has a high recurrence rate. The 2^{nd} line treatment of recurrent cancer is particularly challenging. Current chemotherapeutic treatment at this stage hinges on one decisive point, the platinum-resistance of the tumor. Currently—and insufficiently—platinum-resistance is determined on the basis of clinically determined disease free survival (DFS) after the preceding chemotherapy. Patients with early recurrences after 1^{st} line therapy with carboplatin and paclitaxel or cisplatin and paclitaxel (recurrence occurs within 6 or 12 months after completion of 1^{st} line chemotherapy) are generally not treated in 2^{nd} line with platinum derivates since response rates are less than 25%. Patients with a longer time lapse between initial chemotherapy and recurrence (recurrence occurs after 6 or 12 months of the completion of 1^{st} line therapy) are treated repeatedly with platinum combination therapies as 2^{nd} line, 3^{rd} line therapies.

Standard regimen for recurrent platinum-sensitive disease is carboplatin and paclitaxel, or carboplatin and gemcitabine. Generally, about 25%, 33%, and 60% respond to subsequent platinum-based treatment when the time between last chemotherapy and relapse is 6-12 months, 12-24 months, and greater than 24 months respectively.

### Biological Rational for DNA Methylation Based Biomarkers for Prognosis of Ovarian Carcinoma.

Current research indicates that DNA methylation plays a key role in chemotherapy resistance of ovarian cancer and is therefore suitable for predicting therapy outcome. In the literature, the expression or DNA methylation of the following genes was described to show correlation with treatment outcome (see details below): BRCA/FANCF pathway, ERCC5, EGFR/HER2-neu, and IGF-II.

In addition, genome-wide DNA methylation analyses revealed a correlation between the total number of hypermethylated gene promoters and increased drug resistance (Meng Li et al, Integrative analysis of DNA methylation and gene expression reveals specific signaling pathways associated with platinum resistance in ovarian cancer, BMC Medical Genomics 2009, 2:34 and George S Watts et al, DNA methylation changes in ovarian cancer are cumulative with disease progression and identify tumor stage, BMC Medical Genomics 2008, 1:47). These studies showed that genome-wide microarray approaches can be utilized to identify and characterize novel DNA methylation markers for predicting the prognosis of ovarian cancer (also: Susan H. Wei et al, Prognostic DNA Methylation Biomarkers in Ovarian Cancer, Clin Cancer Res 2006; 12(9))

BRCA/FANCF Pathway.

Women with a mutation (abnormal change) in either the BRCA1 or BRCA2 genes have a significantly increased risk of getting ovarian and breast cancer. For BRCA associated, inherited tumors, it is assumed that the alleles of BRCA1 and BRCA2 are inactivated before tumor development occurs. BRCA1 and BRCA2 are believed to take part in a common pathway involved in maintenance of genomic integrity in cells. Both BRCA proteins have been implicated in important cellular functions, including embryonic development, DNA damage repair, and transcriptional regulation (see Scully and Livingston, Nature 408:429-432, 2000; Zheng et al., Oncogene 19:6159-6175, 2000; Welsh et al., Trends. Genet. 16:69-74, 2000; and MacLachlan et al., J. Biol. Chem. 275:2777-2785, 2000). BRCA1 and BRCA2 have each been implicated in defective homologous recombination DNA repair (see Arvanitis et al., International Journal of Molecular Medicine 10:55-63, 2002), and it is believed that each may be a positive regulator of homologous recombination, with BRCA2 potentially interacting with Rad51, a central homologous recombination effector protein, and BRCA1 regulating GADD45, a DNA damage response gene.

The described genetic defects make women more susceptible to cancer development in these tissues. By age 70, approximately one-half of all women with inherited BRCA1 mutations will get breast and/or ovarian cancer; women with BRCA2 mutations have a comparable risk of breast cancer. Although, women with BRCA mutation show a high probability of getting ovarian cancer they are hypersensitive to platinum based treatment and have a higher chance of overall survival (see Veronesi A et al., BMC Cancer. 5:70, 2005 and Majdak EJ et. al., Cancer. 104(5):1004-12, 2005).

Recent research results show that another mechanism exists for the deactivation of the BRCA genes. In a significant number of sporadic ovarian cancer patients (appr. 12%; see Boyd J. Oncology. 12:399-406, 1998), hypermethylation of the BRCA1 promotor region was observed, causing the gene to be switched off. Clinical studies showed that there is a significant difference in time to progression and overall survival between the two different mechanisms of BRCA deactivation. While patients with BRCA mutation had a median progression free survival of 39.5 months (overall survival 78.6 months), the patients with BRCA methylation had a median progression free survival of 9.8 months (overall survival 35.6 months) (see “BRCA1 promoter methylation predicts adverse ovarian cancer prognosis.” Chiang J W et. al., Gynecol Oncol. 101(3):403-10, 2006).

The mechanism of this phenomenon may be explained the following way. Platinum compounds react with DNA to form adducts leading to the inhibition of DNA replication and transcription, an event essential for its cytotoxic activity. The activity of DNA repair pathways may reverse the effect of platinum compounds, enabling the tumor cell to replicate. While the mutation of the BRCA gene irreversibly silences the repair enzyme, the methylation of the promoter of the gene is a reversible mechanism. The treatment of the cancer cells with platinum compounds puts the cells under selective pressure, enabling those cells to survive that have an unmethylated, ergo functional BRCA repair gene.

Olopade and Wei (in: FANCF methylation contributes to chemoselectivity in ovarian cancer. Cancer Cell. 3(5):417-20, 2003) describe a model of ovarian cancer tumor progression implicates aberrant FANCF promoter methylation that is associated with gene silencing and disruption of the Fanconi-anemia-BRCA pathway. Disruption of the pathway occurs de novo in ovarian cancers and may contribute to selective sensitivity to platinum salts. Similarly D'Andrea (in D'Andrea AD. The Fanconi Anemia/BRCA signalling pathway: disruption in cisplatin-sensitive ovarian cancers. Cell Cycle. 2(4):290-2, 2003) describes that ovarian tumors often exhibit chromosome instability and hypersensitivity to the chemotherapeutic agent cisplatin. This cellular phenotype may result from an acquired disruption of the Fanconi Anemia/BRCA from methylation and silencing of one of the FA genes (FANCF). The serial inactivation and reactivation of the FA/BRCA pathway is described as having important implications for the diagnosis and treatment of ovarian cancers and related cancers. Both these papers describe FANCF methylation.

ERCC5.

ERCC5 is, similar to the genes of the FANCF/BRCA pathway, involved in DNA repair mechanisms. The gene encodes an XPG protein and is key member of the nucleotide excision pathway, the DNA repair mechanism responsible for removing bulky DNA adducts. Similar to the hypothesis described above for BRCA and other genes involved in DNA repair mechanisms, the loss of XPG function is thought to correlate with diminished ability to repair platinum-induced DNA damage, enhancing platinum sensitivity, and prolonging disease free survival. Christine S. Walsh et al. show (Christine S. Walsh et. al. ERCC5 Is a Novel Biomarker of Ovarian Cancer Prognosis, J Clin Oncol 26:2952-2958) on 90 ovarian cancer patients a statistically significant correlation between ERCC5 gene expression and progression-free survival.

EGFR/HER2.

EGFR and HER-2/neu are the most frequently studied molecular biological parameters in epithelial ovarian cancer. The results of these studies were extremely heterogeneous. de Graeff P et al performed a meta analysis of the studies (de Graeff P et al Modest effect of p53, EGFR and HER-2/neu on prognosis in epithelial ovarian cancer: a meta-analysis, Br J. Cancer. 2009 Jul. 7; 101(1):149-59) and concluded that the genes have only a small effect on progression-free survival of ovarian cancer patients and may not be useful for introduction into clinical routine. The hazard ratio for EGFR and HER-2/neu was estimated to be 1.67 and 1.65, respectively.

IGF-II.

The insulin-like growth factor-II (IGF-II) gene has four promoters that produce distinct transcripts which vary by tissue type and developmental stage. Dysregulation of normal promoter usage has been shown to occur in cancer; DNA methylation regulates promoter use. Beeghly A C et al measured the DNA methylation of the IGF-II promoter region on 215 ovarian cancer samples and showed a statistically significant correlation between progression-free survival and the methylation rate of at least one region of the IGF-II promoter (Beeghly A C et al, IGF-II promoter methylation and ovarian cancer prognosis. J Cancer Res Clin Oncol. 2007 October; 133(10):713-23). Beeghly et al. also showed that the methylation rate varies at the different regions of the promoter and that this variation may have biologic implications relating to different clinical features.

Pronounced Need in the Art.

Today, most patients with ovarian cancer are diagnosed with advanced disease (FIGO III and FIGO IV) and will be treated with debulking surgery followed by standard 1^{st} line chemotherapy with carboplatin and paclitaxel or cisplatin and paclitaxel. Prognostic biomarkers based on gene mutations, RNA expression classifiers or methylation markers are not taken into account for therapy decision making. Prognostic molecular markers (biomarkers) that would stratify patients with ovarian cancer into platinum resistant or platinum sensitive patients would enable physicians to use more aggressive therapies e.g. triplet therapies for platinum-resistant patients with poor prognosis, while platinum-sensitive patients could receive standard combinations with carboplatin and paclitaxel or monotherapy of carboplatin or cisplatin.

Biomarkers that can predict either prognosis or response to chemotherapy (the latter being a prognosis after chemotherapy) of individual patients with primary ovarian cancer would enable physicians to better select optimal drug combinations for treatment of primary and recurrent tumors. In particular the prediction of platinum resistance in patients with primary ovarian cancer would enable alternative treatments even in the adjuvant setting. Today, a variety of alternative drugs are available, e.g., taxol inhibits microtubuli formation, gemcitabine inhibits DNA replication, topotecan inhibits the topoisomerase function, doxorubicin functions as antibiotic. In addition, targeted drugs like erlotinib (Tarceva) that block the receptor thyrosine kinase domain of the EGFR (HER1) receptor or bevazicumab (Avastin) that binds the VEGF-A ligand and thus inhibit signaling through the VGFR2(KDR) receptor pathway open new treatment options in combination with standard drugs. Since all these drugs act along different cellular mechanisms they should not display cross-resistances and should be considered as addition to standard chemotherapy, either as third active compound or as maintenance therapy after adjuvant therapy with platinum and taxol.

### DEFINITIONS

The term “methylation status” refers to whether a given cytosine residue in a CpG dinucleotide of a genomic nucleic acid is methylated or unmethylated. The term can also refer to a plurality of CpG dinucleotides within a given genomic nucleic acid.

The term “prognosis” shall be taken to mean a prediction of the likely progression of the disease, in particular response to treatment, aggressiveness and metastatic potential of a tumor. Prognosis may be measured by any variables commonly used in the field, but is most preferably measured by patient survival times.

The term “aggressiveness” is taken to mean one or more of high likelihood of relapse post surgery; below average or below median patient survival; below average or below median disease free survival; below average or below median relapse-free survival; above average tumor-related complications; fast progression of tumor or metastases. Indicators of tumor aggressiveness standard in the art include but are not limited to, tumor stage, tumor grade, nodal status and survival.

The term “survival” shall be taken to include all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis, time of surgery or start of treatment) and end point (e.g. death, recurrence or metastasis).

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The terms “methylation-specific restriction enzymes” or “methylation-sensitive restriction enzymes” shall be taken to mean an enzyme that selectively digests a nucleic acid dependant on the methylation state of its recognition site. Preferred are methylation-specific restriction enzymes, the recognition sequence of which comprises a CG dinucleotide.

Any reference to a gene by name shall be taken to include all transcript variants thereof and all promoter and regulatory elements thereof.

### DESCRIPTION OF THE INVENTION

The present invention provides methods, nucleic acids and kits for prognosis of individual subjects (patients) with ovarian carcinoma. Said method comprises determining the methylation status of at least one target nucleic acid selected from the group consisting of the genes as referred to by name in Table 1 in a biological sample, preferably tissue or blood, isolated from said subject. The target nucleic acid is also referred to as a “marker” herein.

Specifically, the invention refers to a method for prognosis of a subject with ovarian carcinoma on the basis of a biological sample, preferably tissue or blood, that is isolated from the subject and that contains genomic DNA from the carcinoma. The method comprises at least the following steps:

Firstly, the methylation status of at least one CpG dinucleotide in a target sequence, i.e. in a gene or DNA portion, is determined that is selected from the group consisting of the target sequences as referred to by gene name in Table 1, preferably in CLK3, in a biological sample, preferably tissue or blood, isolated from a subject. It is preferred that the target sequence is a target sequence as listed with SEQ ID NOs 1, 6, 11, . . . , 550 in table 1, which are all genomic sequences.

Secondly, from the determined methylation status of the target sequence, the prognosis of the subject with ovarian cancer is deduced. This deduction takes place, depending on the target sequence used, on the basis of the methylation status, i.e. an upmethylation or a downmethylation that was determined for the target sequence, as will be explained in detail below.

When the methylation status of more than one CpG dinucleotide is determined, the determination can be performed on CpG dinucleotides that are located either within the same target sequence or within at least two target sequences selected form the group consisting of the genomic sequences as provided in Table 1.

It is preferred that the methylation status of at least 2, 3, 4, 5, 6, 7, 8, 9, 10, 11, 12, 13, 14, 15, or up to 25 CpG dinucleotides are determined together, preferably of one gene, for determining the prognosis of the subject with ovarian cancer. As pointed out above, the CpG dinucleotides can be located within one target sequence or several target sequences, depending on the number of CpG dinucleotides that are being determined.

The biological sample is preferably selected from the group consisting of ovarian cancer tissue, ovarian tissue, peritoneal tissue, lymph node tissue, fallopian tube tissue, blood, serum, plasma, and peritoneal cavity fluid and combinations thereof. It is preferred that the biological sample stems from ovarian cancer tissue, as this allows the most accurate characterization of the disease.

Specifically, the method preferably comprises isolating genomic DNA from a tumor tissue specimens or blood sample that was obtained from the subject. The subject preferably is a human patient with ovarian cancer.

Preferably, determining the methylation status comprises treating the genomic DNA or a fragment thereof with a chemical reagent or an enzyme containing solution, whereby the base pairing behavior of methylated cytosine bases and/or unmethylated cytosine bases of the nucleic acid are altered such that methylated cytosine bases become distinguishable from unmethylated cytosine bases. This allows for an analysis of the methylation status of the target sequence using common DNA analysis techniques, such as amplification techniques like PCR. The preferred chemical reagent is bisulfite.

Accordingly, the determination of the methylation status of the method preferably comprises amplifying the treated DNA or selected fragments thereof by means of methylation specific primers and/or blocking oligonucleotides. Most preferred, the presence or absence of the amplicons by means of a real-time detection probe is determined.

The invention also refers to a nucleic acid for use in determining of prognosis of ovarian carcinoma in a subject using a biological sample isolated from the subject, or to the use of a nucleic acid for determining prognosis of ovarian carcinoma in a subject using a biological sample isolated from the subject, wherein the nucleic acid is chosen from the group of nucleic acids with the SEQ ID NOs 1 to 550. The invention also refers to the use of such nucleic acids in medicine.

Further, the invention refers to the use of a nucleic acid for determining prognosis of ovarian carcinoma in a subject, wherein the nucleic acid comprises at least 16 contiguous nucleotides of a sequence selected from the group consisting of the bisulfite sequences of Table 1, and sequences complementary thereto.

Further, the invention refers to a nucleic acid for determining prognosis of ovarian carcinoma in a subject that comprises at least 50, preferably 60, 70, or 80 contiguous nucleotides of a sequence selected from the group consisting of the bisulfite sequences as provided in Table 1, and sequences complementary thereto. The use of such nucleic acids for detecting ovarian cancer in a subject is another aspect of the invention.

In one aspect, the invention provides a method comprising the following steps: i) contacting genomic DNA isolated from a biological sample obtained from the subject with at least one reagent, or series of reagents that distinguishes between methylated and non-methylated CpG dinucleotides within at least one target region of the genomic DNA, wherein the nucleotide sequence of said target region comprises at least one CpG dinucleotide sequence from the group consisting of the genes as referred to by name in Table 1 and ii) determining prognosis of ovarian carcinoma. Preferably the target region comprises, or hybridizes under stringent conditions to a sequence of at least 16 contiguous nucleotides of a sequence selected from the group consisting of the genomic sequences as provided in Table 1.

More specifically, the present invention provides a method for determining prognosis of ovarian carcinoma in a subject suitable for use in a diagnostic tool, comprising: obtaining a biological sample comprising genomic nucleic acid(s); contacting the nucleic acid(s), or a fragment thereof, with a reagent or a plurality of reagents sufficient for distinguishing between methylated and non methylated CpG dinucleotide sequences within a target sequence of the subject nucleic acid, wherein the target sequence comprises, or hybridizes under stringent conditions to, a sequence comprising at least 16 contiguous nucleotides of the genomic sequences as provided in Table 1 said contiguous nucleotides comprising at least one CpG dinucleotide sequence; and determining, based at least in part on said distinguishing, the methylation status of at least one target CpG dinucleotide sequence, or an average, or a value reflecting an average methylation status of a plurality of target CpG dinucleotide sequences. The methylation status of a plurality of CpG dinucleotides can for example be determined by numerically averaging hybridization measurements from several contiguous nucleotide sequences comprising at least one CpG dinucleotide sequence, by measuring hybridization of a single contiguous nucleotide sequence comprising more than one CpG dinucleotide sequence, or by requiring concurrent hybridization of several contiguous nucleotide sequences comprising at least one CpG dinucleotide sequence (e.g. MSP MethyLight or HeavyMethyl™ MethyLight assays). It is particularly preferred that the methylation status of a plurality of CpG positions is determined, preferably at least 2, 3, 4, 5, 6, 7, 8, 9, 10, 11, 12, 13, 14, 15 or up to 25 CpG positions.

Further embodiments provide a method for determining prognosis of ovarian carcinoma in a subject (e.g. response prediction to chemotherapy treatment) of ovarian carcinoma, comprising: obtaining a biological sample having subject genomic DNA; extracting the genomic DNA; contacting the genomic DNA, or a fragment thereof, comprising one or more sequences selected from the group consisting of the genomic sequences as provided in Table 1 or a sequence that hybridizes under stringent conditions thereto, with one or more methylation-sensitive restriction enzymes, wherein the genomic DNA of the genomic sequences as provided in Table 1 is either digested thereby to produce digestion fragments, or is not digested thereby; and determining, based on a presence or absence of, or on property of at least one such fragment, the methylation status of at least one CpG dinucleotide sequence of the genomic sequences as provided in Table 1, or more preferably an average, or a value reflecting an average methylation status of a plurality of CpG dinucleotide sequences thereof. Preferably said plurality is at least 2, 5, 10, 15 or 25 CpG positions. Preferably, said enzymatically treated genomic DNA is amplified prior to said determining.

Insofar as the invention refers to response prediction to chemotherapy treatment (i.e. prognosis to chemotherapy treatment), chemotherapy agents are preferably platinum-compounds, such as carboplatin or cisplatin. As described above, such drugs are often given in combination, e.g. together with paclitaxel, gemcitabine, and others like bevazicumab or erlotinib.

Additional embodiments provide novel genomic and chemically modified nucleic acid sequences, as well as oligonucleotides and/or PNA-oligomers for analysis of cytosine methylation patterns within the genomic sequences as provided in Table 1, in particular for a medical use, in particular for determining prognosis of ovarian carcinoma in a subject.

The present invention provides a method for determining prognosis of ovarian carcinoma in a subject comprising determining the methylation levels of at least one gene selected from the group consisting of the genes as referred to by name in Table 1 in a biological sample isolated from said subject. Said markers may be used for determining prognosis of subjects having ovarian cancer.

The present invention provides for the use of the bisulfite technique, in combination with one or more methylation assays, for determination of the methylation status of CpG dinucleotide sequences within the genomic sequences as provided in Table 1. Genomic CpG dinucleotides can be methylated or unmethylated (alternatively known as up- and down-methylated respectively). However the methods of the present invention are suitable for the analysis of biological samples of a heterogeneous nature e.g. a low concentration of tumor cells within a background of stroma tissue or blood. Accordingly, when analyzing the methylation status of a CpG position within such a sample the person skilled in the art may use a quantitative assay for determining the level (e.g. percent, fraction, ratio, proportion or degree) of methylation at a particular CpG position as opposed to a methylation state. Accordingly the term methylation status or methylation state should also be taken to mean a value reflecting the degree of methylation at a CpG position. Unless specifically stated the terms “hypermethylated” or “upmethylated” shall be taken to mean a methylation level above that of a specified cut-off point, wherein said cut-off may be a value representing the average or median methylation level for a given population, or is preferably an optimized cut-off level. The “cut-off” is also referred herein as a “threshold”. In the context of the present invention the terms “methylated”, “hypermethylated” or “upmethylated” shall be taken to include a methylation level of one hundred (100) % (or equivalents thereof) or at or above a selected cut-off methylation value for all CpG positions within and associated with (e.g. in promoter or regulatory regions) at least one gene selected from the group consisting of the genes as referred to by name in Table 1. Unless specifically stated the terms “hypomethylated” or “down-methylated” shall be taken to mean a methylation level below that of a specified cut-off point, wherein said cut-off may be a value representing the average or median methylation level for a given population, or is preferably an optimized cut-off level. The “cut-off” is also referred herein as a “threshold”. In the context of the present invention the terms “unmethylated”, “hypomethylated” or “downmethylated” shall be taken to include a methylation level of zero (0) % (or equivalents thereof) or at or below a selected cut-off methylation value for all CpG positions within and associated with (e.g. in promoter or regulatory regions) at least one gene selected from the group consisting of the genes as referred to by name in Table 1.

According to the present invention, determination of the methylation status of CpG dinucleotide sequences within the genomic sequences as provided in Table 1 have utility in determining the prognosis of ovarian carcinoma patients.

Determining prognosis of ovarian carcinoma in a subject has further utility in the treatment optimization of ovarian cancer patients. More specifically, the most important prognostic factor for ovarian carcinomas after surgery is the response rate to platinum. Therefore it is likely that the markers subject to this invention predict the response to platinum-based chemotherapy.

In one embodiment the invention of said method comprises the following steps: i) contacting genomic DNA (preferably isolated from ovarian tumor tissue; lymph node metastasis tissue; blood; serum; plasma; peritoneal cavity fluid) obtained from the subject with at least one reagent, or series of reagents that distinguishes between methylated and non-methylated CpG dinucleotides within at least one gene selected from the group consisting of the genes as referred to by name in Table 1 (including promoter and regulatory regions thereof) and ii) determining the prognosis of the subject with ovarian carcinoma.

It is preferred that said one or more CpG dinucleotides of at least one gene selected from the group consisting of the genes as referred to by name in Table 1 are comprised of a respective genomic target sequence thereof as provided in the genomic sequences in Table 1 and complements thereof.

In a preferred embodiment, said method comprises the following steps: In the first step, a sample of the tissue to be analyzed is obtained.

The genomic DNA is then isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants and the genomic DNA is then recovered from the solution. The person skilled in the art may also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

Once the nucleic acids have been extracted, the genomic double stranded DNA is analyzed.

In the second step of the method, the genomic DNA sample is treated that methylated cytosines are differentiated form non-methylated cytosines. A number of suitable treatment methods are known in the art, including but not limited to methylation sensitive restriction enzyme digest and treatment of the sample with bisulfite reagents. Both of said methods may be utilized in the method of the present invention, however the bisulfite treatment method is particularly preferred.

### Bisulfite Analysis Method

Methods of bisulfite treatment are known in the art. The bisulfite treated DNA is preferably purified prior to the subsequent method steps. This may be conducted by any means known in the art, such as but not limited to ultrafiltration. The purification is carried out according to the manufacturer's protocol. Suitable kits are commercially available, such as but not limited to the EpiTect Bisulfite Kit™ and EZ DNA Methylation Kit™.

In the third step of the method, fragments of the treated DNA are amplified, using one or more pairs of primer oligonucleotides according to the present invention, and an amplification enzyme. Preferably, the amplification is carried out by means of a polymerase chain reaction (PCR), in one embodiment said amplicons are 80 to 1,000 base pairs in length. Preferably each of said pair of primer oligonucleotides includes at least two oligonucleotides whose sequences are each reverse complementary, identical, or hybridize under stringent or highly stringent conditions to an at least 16-base-pair long segment of the base sequences of a sequence selected from the group consisting of the bisulfite sequences as provided in Table 1 and sequences complementary thereto.

Alternatively, the methylation status of pre-selected CpG positions within at least one gene selected from the group consisting of the genes as referred to by name in Table 1 and preferably within a target nucleic acid sequences thereof according to the genomic sequences in Table 1 may be detected by use of methylation-specific primer oligonucleotides. Said technique has been described in U.S. Pat. No. 6,265,171 (hereby incorporated by reference in its entirety). MSP primer pairs comprise at least one primer which hybridizes to a bisulfite treated CpG dinucleotide. Therefore, the sequence of said primers comprises at least one CpG dinucleotide. MSP primers specific for methylated DNA contain a “C” or “G” at the position of the C position in the CpG. MSP primers specific for non-methylated DNA contain a “T” or “A” at the position of the C position in the CpG. Preferably, therefore, the base sequence of said primers is required to comprise a sequence having a length of at least 9 nucleotides which hybridizes to a treated nucleic acid sequence according to one of the bisulfite sequences as provided in Table 1 and sequences complementary thereto, wherein the base sequence of said oligomers comprises at least one CpG dinucleotide.

A further preferred embodiment of the method comprises the use of blocker oligonucleotides (the HeavyMethyl™ assay). The use of such blocker oligonucleotides has been described by Yu et al., BioTechniques 23:714-720, 1997. Blocking probe oligonucleotides are hybridised to the bisulfite treated nucleic acid in tandem with the PCR primers. PCR amplification of the nucleic acid is terminated at the 5′ position of the blocking probe, such that amplification of a nucleic acid is suppressed where the complementary sequence to the blocking probe is present. Disruption of polymerase-mediated amplification requires that blocker oligonucleotides are not elongated by the polymerase. This is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Said probes are preferably designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpA’ or ‘TpG’ at the position in question, as opposed to a ‘CpG’ if the suppression of amplification of methylated nucleic acids is desired.

It is further preferred that polymerase-mediated decomposition of the blocker oligonucleotides should be minimized. This may be achieved by use of a polymerase lacking 5′-3′ exonuclease activity, or use of modified blocker oligonucleotides having, for example, thioate bridges at the 5′-terminii thereof that render the blocker molecule nuclease-resistant.

Preferably, the base sequence of said blocking oligonucleotides is required to comprise a sequence having a length of at least 9 nucleotides which hybridizes to a treated nucleic acid sequence according to one of the bisulfite sequences as provided in Table 1 and sequences complementary thereto, wherein the base sequence of said oligonucleotides comprises at least one CpG, TpG or CpA dinucleotide. More preferably the base sequence of said blocking oligonucleotides is required to comprise a sequence having a length of at least 9 nucleotides which hybridizes to a treated nucleic acid sequence selected from the group consisting of the 0% methylated bisulfite sequences as provided in Table 1 and sequences complementary thereto.

The nucleic acid amplicons may be detectably labeled. Any suitable labels known in the art may be utilized, including but not limited to fluorescence labels, radionuclides, or detachable molecule fragments having a typical mass, which renders the detectable in a mass spectrometer.

In the fourth step of the method, the amplicons obtained during the third step of the method are analyzed in order to ascertain the methylation status of said at least one or more CpG dinucleotides prior to the treatment.

In embodiments where the amplicons were obtained by means of MSP amplification, the presence or absence of an amplicon is in itself indicative of the methylation state of the CpG positions covered by the primer, according to the base sequences of said primer. The presence or absence of said amplicon may be detected by means of any suitable means known in the art, including the detection of suitable labels, or by means of a nucleic acid detection probe.

Amplicons obtained by means of both standard and methylation specific PCR may be further analyzed by means of methods such as, but not limited to, array technology and probe based technologies as well as by means of techniques such as sequencing and template directed extension.

In one embodiment of the method, the amplicons synthesized in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplicons serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG, TpG or CpA dinucleotide. The hybridizing portion of the hybridizing nucleic acids is typically at least 9, 15, 20, 25, 30 or 35 nucleotides in length. However, longer molecules have inventive utility, and are thus within the scope of the present invention.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within a sequence selected from the group consisting of the genomic sequences as provided in Table 1, and the equivalent positions within the bisulfite sequences as provided in Table 1. Said oligonucleotides may also be peptide nucleic acids. The non-hybridized amplicons are then removed, and the hybridized amplicons are detected. It is preferred that labels attached to the amplicons are identifiable at each position of the solid phase at which an oligonucleotide sequence is located.

In yet a further embodiment of the method, the genomic methylation status of the CpG positions may be ascertained by means of oligonucleotide probes (as detailed above) that are hybridized to the bisulfite treated DNA in tandem with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a non-extendible interrogating oligonucleotide, called a TaqMan™ probe, which, in preferred embodiments, is designed to hybridize to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethyLightTM™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

In a further preferred embodiment of the method, the fourth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fourth step of the method comprises sequencing and subsequent sequence analysis of the amplicon generated in the third step of the method (Sanger F., et al., Proc Natl Acad Sci USA 74:5463-5467, 1977).

In the most preferred embodiment of the method the genomic nucleic acids are isolated and treated according to the first three steps of the method outlined above, namely:
a) Obtaining a tumor tissue or blood sample from a patient with ovarian cancer;
b) Extracting or otherwise isolating said genomic DNA from the tumor tissue or blood sample;
c) Treating the tumor DNA of b), or a fragment thereof, with one or more reagents e.g. bisulfite to convert cytosine bases that are unmethylated in the 5-position thereof to uracil or to another base that is detectably dissimilar to cytosine in terms of hybridization properties; and
d) Amplifying selected fragments of said treated DNA by means of methylation specific primers and/or blocking oligonucleotides, and
e) Determining presence or absence of said amplicons by means of a real-time detection probe, as described above.

Preferably, where the subsequent amplification of d) is carried out by means of methylation specific primers, as described above, said methylation specific primers comprise a sequence having a length of at least 9 nucleotides which hybridizes to a treated nucleic acid sequence according to one of the bisulfite sequences as provided in Table 1 and sequences complementary thereto, wherein the base sequence of said oligomers comprise at least one CpG dinucleotide.

Step e) of the method, namely the detection of the specific amplicons indicative of the methylation status of one or more CpG positions according to the genomic sequences as provided in Table 1 is carried out by means of real-time detection methods as described above.

### Restriction Enzyme Analysis

Additional embodiments of the invention provide a method for the analysis of the methylation status of the at least one gene selected from the group consisting of the genes as referred to by name in Table 1 (preferably the genomic sequences as provided in Table 1, and complements thereof) without the need for bisulfite conversion. Methods are known in the art wherein a methylation sensitive restriction enzyme reagent, or a series of restriction enzyme reagents comprising methylation sensitive restriction enzyme reagents that distinguishes between methylated and non-methylated CpG dinucleotides within a target region are utilized in determining methylation.

In the third step, the DNA (or fragments thereof) is digested with one or more methylation sensitive restriction enzymes. The digestion is carried out such that hydrolysis of the DNA at the restriction site is informative of the methylation status of a specific CpG dinucleotide of at least one gene selected from the group consisting of the genes as referred to by name in Table 1, it is particularly preferred that a target region of said gene(s) selected from Table 1 is analyzed.

Preferably, the methylation-specific restriction enzyme is selected from the group consisting of Bsi E1, Hga I HinPl, Hpy99I, Ava I, Bce AI, Bsa HI, Bisl, BstUI, BshI236I, AccII, BstFNI, McrBC, GlaI, MvnI, HpaII (HapII), HhaI, AciI, SmaI, HinP1I, HpyCH4IV, EagI and mixtures of two or more of the above enzymes. Preferred is a mixture containing the restriction enzymes BstUI, HpaII, HpyCH41V and HinP1I.

In the fourth step, which is optional but a preferred embodiment, the restriction fragments are amplified. This is preferably carried out using a polymerase chain reaction, and said amplicons may carry suitable detectable labels as discussed above, namely fluorophore labels, radionuclides and mass labels. Particularly preferred is amplification by means of an amplification enzyme and one or more primer pairs, each member of said pair comprising, in each case a contiguous sequence at least 16 nucleotides in length that is complementary to, or hybridizes under moderately stringent or stringent conditions to a sequence selected from the group consisting of the genomic sequences as provided in Table 1, and complements thereof. Preferably said contiguous sequence is at least 16, 20 or 25 nucleotides in length. In an alternative embodiment said primers may be complementary to any adaptors linked to the fragments.

In the fifth step the amplificons are detected. The detection may be by any means standard in the art, for example, but not limited to, gel electrophoresis analysis, hybridization analysis, incorporation of detectable tags within the PCR products, DNA array analysis, MALDI or ESI analysis. Preferably said detection is carried out by hybridization to at least one nucleic acid or peptide nucleic acid comprising in each case a contiguous sequence at least 16 nucleotides in length that is complementary to, or hybridizes under moderately stringent or stringent conditions to a sequence selected from the group consisting of the genomic sequences as provided in Table 1, and complements thereof. Preferably said contiguous sequence is at least 16, 20 or 25 nucleotides in length.

Subsequent to the determination of the methylation state or level of the genomic nucleic acids the prognosis of said subject having ovarian carcinoma is deduced based upon the methylation state or level of at least one CpG dinucleotide sequence of the genomic sequences as provided in Table 1, or a weighted average, a wighed geometric average, or a ranked average, or a arithmetic combination of logistic functions of methylation states or levels or a value mathematically reflecting a combined methylation state of a plurality of CpG dinucleotide sequences of the genomic sequences as provided in Table 1, wherein methylation is associated with patient prognosis.

Wherein said methylation is determined by quantitative means and for markers whose hypermethylation is associated with poor prognosis the cut-off point for determining said prognosis is preferably close to 0% (i.e. wherein a sample displays any degree of methylation it is determined as having a poor prognosis). For markers whose hypomethylation is associated with poor prognosis the cut-off point for determining said prognosis is preferably close to 100% (i.e. wherein a sample displays any degree of unmethylated DNA it is determined as having a poor prognosis).

Another preferred way of identifying the cut-off values is based on the distribution of measurements collected on all 90 tumor samples (see section “Cancer Samples” above), or any set of tumor or blood samples that stems from a representative collective of ovarian cancer patients. A certain quantile of this distribution is chosen as the cut-off point, preferably 50% (the median) but also values ranging from 25% through 75% or from 10% to 90%. For example, the 55% quantile would be that value for which 55% of all measurements are below that value and 45% are above that value. For genes whose hypomethylation is indicative of poor patient prognosis, a measurement that is below the cut-off would indicate a poor prognosis. For genes whose hypermethylation is indicative of poor patient prognosis, a measurement above the cut-off would indicate a poor prognosis.

Nonetheless, it is foreseen that the person skilled in the art may wish to adjust said cut-off value in order to provide an assay of a particularly preferred sensitivity or specificity. Accordingly said cut-off value may be increased (thus increasing the specificity) for genes whose hypermethylation is indicative of poor patient prognosis. Said cut off value may be within a range selected form the group consisting of 0%-5%, 5%-10%, 10%-15%, 15%-20%, 20%-30% and 30%-50%. Particularly preferred are the cut-offs 0.01%, 0.1%, 1% and 10%. Said cut-off value may be decreased (thus increasing the specificity) for genes whose hypomethylation is indicative of poor patient prognosis. Said cut off value may be within a range selected form the group consisting of 100%-95%, 95%-90%, 90%-85%, 85%-80%, 80%-70% and 70%-50%. Particularly preferred are the cut-offs 99.99%, 99.90%, 99% and 90%.

Another preferred determination to determine the cut-off value is based on the distribution of measurements taken from a sampling of ovarian cancer tissues that is representative of the target population. Preferably, this sampling is the set of cancer samples described in the section “Patient Samples” above. A certain quantile is chosen as the cut-off point, preferably 50% but also values ranging from 25% through 75%. For genes whose hypomethylation is indicative of poor patient prognosis, a measurement below the threshold would indicate a poor prognosis. For genes whose hypermethylation is indicative of poor patient prognosis, a measurement above the threshold would indicate a poor prognosis. The distribution of measurements with respect to which the threshold is determined as a quantile can be a group of patients.

Upon determination of the methylation of at least one gene selected from the group consisting of the genes as referred to by name in Table 1 the prognosis of said ovarian carcinoma patient is determined.

For the following genes, methylation and/or hypermethylation is associated with poor prognosis:

CLK3, MTMR4, NFE2L1, PERLD1, AKAP2, ANAPC1, ANKRD47, ATF7, ATP5G1, BCL2 (genomic target regions SEQ ID NO: 66, 71), C9orf3, COIL, CSF2, E2F3, ELMO2, ERBB2, GABRG2, GPC5, KIAA0100, KISS1, MARCH3, MRO, MSX1, NCOA6, PDGFRA, RPL23A (genomic target regions SEQ ID NO: 471, 476), RUFY3, TP53, TSPYL1, UBXD3, ZNF420

In a further embodiment, an increased methylation level of these genes is correlated to poor prognosis. In said embodiment the level of methylation is inversely proportional to patient survival time.

For the following genes, absence of methylation and/or hypomethylation is associated with poor prognosis:

BCL2 (genomic target regions SEQ ID NO: 76, 81), DMP1, HSPA6, PAPOLA, RPL23A (genomic target regions SEQ ID NO: 481, 486), SOX3.

In a further embodiment, a decreased level of methylation is correlated to poor prognosis. In said embodiment the level of methylation is proportional to patient survival time.

### Nucleic Acids

The disclosed invention provides treated nucleic acids the bisulfite sequences as provided in Table 1, derived from the genomic sequences as provided in Table 1. The sequences of the bisulfite sequences as provided in Table 1 are non-naturally occurring sequences and provide the sequences of various bisulfite modified nucleic acids of the genomic sequences as provided in Table 1.

In a preferred embodiment of the invention, the invention provides a non-naturally occurring modified nucleic acid comprising a sequence of at least 16 contiguous nucleotide bases in length of a sequence selected from the group consisting of the bisulfite sequences as provided in Table 1. In further preferred embodiments of the invention said nucleic acid is at least 50, 100, 150, 200, 250 or 500 base pairs in length of a segment of the nucleic acid sequence disclosed in the bisulfite sequences as provided in Table 1. Particularly preferred is a nucleic acid molecule that is identical or complementary to all or a portion of the bisulfite sequences as provided in Table 1 but not the genomic sequences as provided in Table 1 or other naturally occurring DNA.

It is preferred that said sequence comprises at least one CpG, TpG or CpA dinucleotide and sequences complementary thereto. The sequences of the bisulfite sequences of Table 1 provide non-naturally occurring modified versions of the nucleic acid according to the genomic sequences of Table 1, wherein the modification of each genomic sequence results in the synthesis of a nucleic acid having a sequence that is unique and distinct from said genomic sequence as follows. For each sense strand of genomic DNA, e.g., the genomic sequences as provided in Table 1, four converted versions are disclosed. A first version wherein “C” is converted to “T,” but “CpG” remains “CpG” (i.e., corresponds to case where, for the genomic sequence, 100% “C” residues of CpG dinucleotide sequences are methylated and are thus not converted); a second version of each genomic sequences is provided, wherein “C” is converted to “T” for all “C” residues, including those of “CpG” dinucleotide sequences (i.e., corresponds to case where, for the genomic sequences, 100% of “C” residues of CpG dinucleotide sequences are unmethylated). A third chemically converted version discloses the complement of the disclosed genomic DNA sequence (i.e. antisense strand), wherein “C” is converted to “T,” but “CpG” remains “CpG” (i.e., corresponds to case where 100% of “C” residues of CpG dinucleotide sequences are methylated and are thus not converted); a final bisulfite converted version of each sequence, discloses the complement of the disclosed genomic DNA sequence (i.e. antisense strand), wherein “C” is converted to “T” for 100% of “C” residues, including those of “CpG” dinucleotide sequences (i.e., corresponds to case where, for the complement (antisense strand) of each genomic sequence, all “C” residues of CpG dinucleotide sequences are unmethylated).

Alternatively, the invention further provides oligonucleotides or oligomers suitable for use in the methods of the invention for detecting the cytosine methylation state within genomic or treated (bisulfite modified) DNA, according to SEQ ID Nos: 1 to 550. Accordingly, said oligonucleotide or oligomer nucleic acids provide novel diagnostic means. Said oligonucleotide or oligomer comprise a nucleic acid sequence having a length of at least nine (9) nucleotides which is identical to, hybridizes, under moderately stringent or stringent conditions (as defined herein above), to a treated nucleic acid sequence according to the bisulfite sequences as provided in Table 1 and/or sequences complementary thereto, or to a genomic sequence according to the genomic sequences as provided in Table 1 and/or sequences complementary thereto.

Particularly preferred is a nucleic acid molecule that hybridizes under moderately stringent and/or stringent hybridization conditions to all or a portion of the sequences the bisulfite sequences as provided in Table 1, but not the genomic sequences as provided in Table 1 or other human genomic DNA.

The identical or hybridizing portion of the hybridizing nucleic acids is typically at least 9, 16, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NOs: 1 to 550, or to the complements thereof.

In further embodiments, the present invention provides a set of at least two (2) oligonucleotides that are used as ‘primer’ oligonucleotides for amplifying DNA sequences of one of SEQ ID NOs: 1 to 550 and sequences complementary thereto, or segments thereof.

It is particularly preferred that the oligomers according to the invention are utilized for determining the prognosis of ovarian carcinoma patients.

Genomic SEQ ID NO: 1 provides the preferred target genomic nucleic acid of the gene CLK3. Furthermore, SEQ ID NO: 6, 11 each provide a particularly preferred target region thereof.

Genomic SEQ ID NO: 16 provides the preferred target genomic nucleic acid of the gene MTMR4. Furthermore, SEQ ID NO: 21, 26, 31, 36 each provide a particularly preferred target region thereof.

Genomic SEQ ID NO: 41 provides the preferred target genomic nucleic acid of the gene NFE2L1. Furthermore, SEQ ID NO: 46, 51, 56, 61 each provide a particularly preferred target region thereof.

Genomic SEQ ID NO: 66 provides the preferred target genomic nucleic acid of the gene PERLD1. Furthermore, SEQ ID NO: 71, 76, 81, 86, 91, 96, 101, 106, 111 each provide a particularly preferred target region thereof.

Genomic SEQ ID NO: 116 provides the preferred target genomic nucleic acid of the gene AKAP2. Furthermore, SEQ ID NO: 121, 126, 131 each provide a particularly preferred target region thereof.

Genomic SEQ ID NO: 136 provides the preferred target genomic nucleic acid of the gene ANAPC1. Furthermore, SEQ ID NO: 141 provides a particularly preferred target region thereof.

Genomic SEQ ID NO: 146 provides the preferred target genomic nucleic acid of the gene ANKRD47. Furthermore, SEQ ID NO: 151, 156 each provide a particularly preferred target region thereof.

Genomic SEQ ID NO: 161 provides the preferred target genomic nucleic acid of the gene ATF7. Furthermore, SEQ ID NO: 166 provides a particularly preferred target region thereof.

Genomic SEQ ID NO: 171 provides the preferred target genomic nucleic acid of the gene ATP5G1. Furthermore, SEQ ID NO: 176 provides a particularly preferred target region thereof.

Genomic SEQ ID NO: 181 provides the preferred target genomic nucleic acid of the gene BCL2. Furthermore, SEQ ID NO: 186, 191, 196 each provide a particularly preferred target region thereof.

Genomic SEQ ID NO: 201 provides the preferred target genomic nucleic acid of the gene C9orf3. Furthermore, SEQ ID NO: 206 provides a particularly preferred target region thereof.

Genomic SEQ ID NO: 211 provides the preferred target genomic nucleic acid of the gene COIL. Furthermore, SEQ ID NO: 216 provides a particularly preferred target region thereof.

Genomic SEQ ID NO: 221 provides the preferred target genomic nucleic acid of the gene CSF2. Furthermore, SEQ ID NO: 226 provides a particularly preferred target region thereof.

Genomic SEQ ID NO: 231 provides the preferred target genomic nucleic acid of the gene DMP 1. Furthermore, SEQ ID NO: 236 provides a particularly preferred target region thereof.

Genomic SEQ ID NO: 241 provides the preferred target genomic nucleic acid of the gene E2F3. Furthermore, SEQ ID NO: 246, 251 each provide a particularly preferred target region thereof.

Genomic SEQ ID NO: 256 provides the preferred target genomic nucleic acid of the gene ELMO2. Furthermore, SEQ ID NO: 261 provides a particularly preferred target region thereof.

Genomic SEQ ID NO: 266 provides the preferred target genomic nucleic acid of the gene ERBB2. Furthermore, SEQ ID NO: 271, 276, 281, 286 each provide a particularly preferred target region thereof.

Genomic SEQ ID NO: 291 provides the preferred target genomic nucleic acid of the gene GABRG2. Furthermore, SEQ ID NO: 296 provides a particularly preferred target region thereof.

Genomic SEQ ID NO: 301 provides the preferred target genomic nucleic acid of the gene GPC5. Furthermore, SEQ ID NO: 306, 311, 316 each provide a particularly preferred target region thereof.

Genomic SEQ ID NO: 321 provides the preferred target genomic nucleic acid of the gene HSPA6. Furthermore, SEQ ID NO: 326 provides a particularly preferred target region thereof.

Genomic SEQ ID NO: 331 provides the preferred target genomic nucleic acid of the gene KIAA0100. Furthermore, SEQ ID NO: 336 provides a particularly preferred target region thereof.

Genomic SEQ ID NO: 341 provides the preferred target genomic nucleic acid of the gene KISS1. Furthermore, SEQ ID NO: 346, 351 each provide a particularly preferred target region thereof.

Genomic SEQ ID NO: 356 provides the preferred target genomic nucleic acid of the gene MARCH3. Furthermore, SEQ ID NO: 361 provides a particularly preferred target region thereof.

Genomic SEQ ID NO: 366 provides the preferred target genomic nucleic acid of the gene MRO. Furthermore, SEQ ID NO: 371 provides a particularly preferred target region thereof.

Genomic SEQ ID NO: 376 provides the preferred target genomic nucleic acid of the gene MSX1. Furthermore, SEQ ID NO: 381, 386, 391, 396, 401, 406, 411, 416, 421, 426, 431 each provide a particularly preferred target region thereof.

Genomic SEQ ID NO: 436 provides the preferred target genomic nucleic acid of the gene NCOA6. Furthermore, SEQ ID NO: 441 provides a particularly preferred target region thereof.

Genomic SEQ ID NO: 446 provides the preferred target genomic nucleic acid of the gene PAPOLA. Furthermore, SEQ ID NO: 451 provides a particularly preferred target region thereof.

Genomic SEQ ID NO: 456 provides the preferred target genomic nucleic acid of the gene PDGFRA. Furthermore, SEQ ID NO: 461, 466 each provide a particularly preferred target region thereof.

Genomic SEQ ID NO: 471 provides the preferred target genomic nucleic acid of the gene RPL23A. Furthermore, SEQ ID NO: 476, 481, 486 each provide a particularly preferred target region thereof.

Genomic SEQ ID NO: 491 provides the preferred target genomic nucleic acid of the gene RUFY3. Furthermore, SEQ ID NO: 496 provides a particularly preferred target region thereof.

Genomic SEQ ID NO: 501 provides the preferred target genomic nucleic acid of the gene SOX3. Furthermore, SEQ ID NO: 506 provides a particularly preferred target region thereof.

Genomic SEQ ID NO: 511 provides the preferred target genomic nucleic acid of the gene TP53. Furthermore, SEQ ID NO: 516 provides a particularly preferred target region thereof.

Genomic SEQ ID NO: 521 provides the preferred target genomic nucleic acid of the gene TSPYL 1. Furthermore, SEQ ID NO: 526 provides a particularly preferred target region thereof.

Genomic SEQ ID NO: 531 provides the preferred target genomic nucleic acid of the gene UBXD3. Furthermore, SEQ ID NO: 536 provides a particularly preferred target region thereof.

Genomic SEQ ID NO: 541 provides the preferred target genomic nucleic acid of the gene ZNF420. Furthermore, SEQ ID NO: 546 provides a particularly preferred target region thereof.

### Kits

Moreover, an additional aspect of the present invention is a kit comprising: a means for determining methylation of at least one gene selected from the group consisting of the genes as referred to by name in Table 1. The means for determining methylation of at least one gene selected from the group consisting of the genes as referred to by name in Table 1 comprise preferably a bisulfite-containing reagent; one or a plurality of oligonucleotides consisting whose sequences in each case are identical, are complementary, or hybridize under stringent or highly stringent conditions to a 9 or more preferably 18 base long segment of a sequence selected from the bisulfite sequences as provided in Table 1; and optionally instructions for carrying out and evaluating the described method of methylation analysis. In one embodiment the base sequence of said oligonucleotides comprises at least one CpG, CpA or TpG dinucleotide.

In a further embodiment, said kit may further comprise standard reagents for performing a CpG position-specific methylation analysis, wherein said analysis comprises one or more of the following techniques: MS-SNuPE, MSP, MethyLight™, HeavyMethyl, COBRA, and nucleic acid sequencing. However, a kit of the present invention can also contain only part of the aforementioned components.

In a preferred embodiment the kit may comprise additional bisulfite conversion reagents selected from the group consisting: DNA denaturation buffer; sulfonation buffer; DNA recovery reagents or kits (e.g., precipitation, ultrafiltration, affinity column); desulfonation buffer; and DNA recovery components.

In a further alternative embodiment, the kit may contain further elements packaged in separate containers, such as but not limited to a polymerase and a reaction buffer optimized for primer extension mediated by the polymerase, such as PCR. In another embodiment of the invention the kit further comprises means for obtaining a biological sample of the patient. Preferred is a kit, which further comprises a container suitable for containing the means for determining methylation of at least one gene selected from the group consisting of the genes as referred to by name in Table 1 in the biological sample of the patient, and most preferably further comprises instructions for use and interpretation of the kit results. In a preferred embodiment the kit comprises: (a) a bisulfite reagent; (b) a container suitable for containing the said bisulfite reagent and the biological sample of the patient; (c) at least one set of primer oligonucleotides containing two oligonucleotides whose sequences in each case are identical, are complementary, or hybridize under stringent or highly stringent conditions to an at least 9 or more preferably 18 base long segment of a sequence selected from the bisulfite sequences as provided in Table 1; and optionally (d) instructions for use and interpretation of the kit results. In an alternative preferred embodiment the kit comprises: (a) a bisulfite reagent; (b) a container suitable for containing the said bisulfite reagent and the biological sample of the patient; (c) at least one oligonucleotides and/or PNA-oligomer having a length of at least 9 or 18 nucleotides which is identical to or hybridizes to a bisulfite treated nucleic acid sequence according to one of the bisulfite sequences as provided in Table 1 and sequences complementary thereto; and optionally (d) instructions for use and interpretation of the kit results.

In an alternative embodiment the kit comprises: (a) a bisulfite reagent; (b) a container suitable for containing the said bisulfite reagent and the biological sample of the patient; (c) at least one set of primer oligonucleotides containing two oligonucleotides whose sequences in each case are identical, are complementary, or hybridize under stringent or highly stringent conditions to an at least 9 or more preferably 18 base long segment of a sequence selected from the bisulfite sequences as provided in Table 1; (d) at least one oligonucleotides and/or PNA-oligomer having a length of at least 9 or 16 nucleotides which is identical to or hybridizes to a pre-treated nucleic acid sequence according to one of the bisulfite sequences as provided in Table 1 and sequences complementary thereto; and optionally (e) instructions for use and interpretation of the kit results.

The kit may also contain other components such as buffers or solutions suitable for blocking, washing or coating, packaged in a separate container.

Moreover, an additional aspect of the present invention is an alternative kit comprising a means for determining the CpG methylation status of at least one gene selected from the group consisting of the genes as referred to by name in Table 1, wherein said means comprise preferably at least one methylation specific restriction enzyme; one or a plurality of primer oligonucleotides (preferably one or a plurality of primer pairs) suitable for the amplification of a sequence comprising at least one CpG dinucleotide of a sequence selected from the genomic sequences as provided in Table 1; and optionally instructions for carrying out and evaluating the described method of methylation analysis. In one embodiment the base sequence of said oligonucleotides are identical, are complementary, or hybridize under stringent or highly stringent conditions to an at least 18 base long segment of a sequence selected from the genomic sequences as provided in Table 1.

In a further embodiment said kit may comprise one or a plurality of oligonucleotide probes for the analysis of the digest fragments, preferably said oligonucleotides are identical, are complementary, or hybridize under stringent or highly stringent conditions to an at least 16 base long segment of a sequence selected from the genomic sequences as provided in Table 1, it is further preferred that the sequence thereof comprises at least one or more CpG dinucleotides.

In a preferred embodiment the kit may comprise additional reagents selected from the group consisting: buffer (e.g. restriction enzyme, PCR, storage or washing buffers); DNA recovery reagents or kits (e.g., precipitation, ultrafiltration, affinity column) and DNA recovery components.

In a further alternative embodiment, the kit may contain, packaged in separate containers, a polymerase and a reaction buffer optimized for primer extension mediated by the polymerase, such as PCR. In another embodiment of the invention the kit further comprising means for obtaining a biological sample of the patient. In a preferred embodiment the kit comprises: (a) a methylation sensitive restriction enzyme reagent; (b) a container suitable for containing the said reagent and the biological sample of the patient; (c) at least one set of oligonucleotides one or a plurality of nucleic acids or peptide nucleic acids which are identical, are complementary, or hybridise under stringent or highly stringent conditions to an at least 9 base long segment of a sequence selected from the genomic sequences as provided in Table 1; and optionally (d) instructions for use and interpretation of the kit results.

In an alternative preferred embodiment the kit comprises: (a) a methylation sensitive restriction enzyme reagent; (b) a container suitable for containing the said reagent and the biological sample of the patient; (c) at least one set of primer oligonucleotides suitable for the amplification of a sequence comprising at least one CpG dinucleotide of a sequence selected from the genomic sequences as provided in Table 1; and optionally (d) instructions for use and interpretation of the kit results.

In an alternative embodiment the kit comprises: (a) a methylation sensitive restriction enzyme reagent; (b) a container suitable for containing the said reagent and the biological sample of the patient; (c) at least one set of primer oligonucleotides suitable for the amplification of a sequence comprising at least one CpG dinucleotide of a sequence selected from the genomic sequences as provided in Table 1; (d) at least one set of oligonucleotides one or a plurality of nucleic acids or peptide nucleic acids which are identical, are complementary, or hybridise under stringent or highly stringent conditions to an at least 9 base long segment of a sequence selected from the genomic sequences as provided in Table 1 and optionally (e) instructions for use and interpretation of the kit results.

The kit may also contain other components such as buffers or solutions suitable for blocking, washing or coating, packaged in a separate container.

TABLE 1
Target sequences according to the invention are shown that can be used as a marker to determine the prognosis of
ovarian carcinoma in a subject. The genomic sequence of each target sequence can be used in the method of the invention
to determine the prognosis of ovarian carcinoma in a subject. The corresponding bisulfite sequences i.e. the sequences
obtainable from the respective target sequence after treatment with bisulfite (when the genomic target sequence
is either 100% or 0% methylated) can be used in determining the prognosis of ovarian carcinoma in a subject according
to the invention. In the left column, target sequences are referred to by gene name.

| | | Bisulfite Sequences | | | |
|---|---|---|---|---|---|
| | | 100% methylated | 0% methylated | 100% methylated | 0% methylated |
| | Genomic | bisulfite converted | bisulfite converted | bisulfite converted | bisulfite converted |
| GeneName | Sequence | sense strand | sense strand | antisense strand | antisense strand |
| CLK3 | SEQ ID NO 1 | SEQ ID NO 2 | SEQ ID NO 3 | SEQ ID NO 4 | SEQ ID NO 5 |
| CLK3 | SEQ ID NO 6 | SEQ ID NO 7 | SEQ ID NO 8 | SEQ ID NO 9 | SEQ ID NO 10 |
| CLK3 | SEQ ID NO 11 | SEQ ID NO 12 | SEQ ID NO 13 | SEQ ID NO 14 | SEQ ID NO 15 |
| MTMR4 | SEQ ID NO 16 | SEQ ID NO 17 | SEQ ID NO 18 | SEQ ID NO 19 | SEQ ID NO 20 |
| MTMR4 | SEQ ID NO 21 | SEQ ID NO 22 | SEQ ID NO 23 | SEQ ID NO 24 | SEQ ID NO 25 |
| MTMR4 | SEQ ID NO 26 | SEQ ID NO 27 | SEQ ID NO 28 | SEQ ID NO 29 | SEQ ID NO 30 |
| MTMR4 | SEQ ID NO 31 | SEQ ID NO 32 | SEQ ID NO 33 | SEQ ID NO 34 | SEQ ID NO 35 |
| MTMR4 | SEQ ID NO 36 | SEQ ID NO 37 | SEQ ID NO 38 | SEQ ID NO 39 | SEQ ID NO 40 |
| NFE2L1 | SEQ ID NO 41 | SEQ ID NO 42 | SEQ ID NO 43 | SEQ ID NO 44 | SEQ ID NO 45 |
| NFE2L1 | SEQ ID NO 46 | SEQ ID NO 47 | SEQ ID NO 48 | SEQ ID NO 49 | SEQ ID NO 50 |
| NFE2L1 | SEQ ID NO 51 | SEQ ID NO 52 | SEQ ID NO 53 | SEQ ID NO 54 | SEQ ID NO 55 |
| NFE2L1 | SEQ ID NO 56 | SEQ ID NO 57 | SEQ ID NO 58 | SEQ ID NO 59 | SEQ ID NO 60 |
| NFE2L1 | SEQ ID NO 61 | SEQ ID NO 62 | SEQ ID NO 63 | SEQ ID NO 64 | SEQ ID NO 65 |
| PERLD1 | SEQ ID NO 66 | SEQ ID NO 67 | SEQ ID NO 68 | SEQ ID NO 69 | SEQ ID NO 70 |
| PERLD1 | SEQ ID NO 71 | SEQ ID NO 72 | SEQ ID NO 73 | SEQ ID NO 74 | SEQ ID NO 75 |
| PERLD1 | SEQ ID NO 76 | SEQ ID NO 77 | SEQ ID NO 78 | SEQ ID NO 79 | SEQ ID NO 80 |
| PERLD1 | SEQ ID NO 81 | SEQ ID NO 82 | SEQ ID NO 83 | SEQ ID NO 84 | SEQ ID NO 85 |
| PERLD1 | SEQ ID NO 86 | SEQ ID NO 87 | SEQ ID NO 88 | SEQ ID NO 89 | SEQ ID NO 90 |
| PERLD1 | SEQ ID NO 91 | SEQ ID NO 92 | SEQ ID NO 93 | SEQ ID NO 94 | SEQ ID NO 95 |
| PERLD1 | SEQ ID NO 96 | SEQ ID NO 97 | SEQ ID NO 98 | SEQ ID NO 99 | SEQ ID NO 100 |
| PERLD1 | SEQ ID NO 101 | SEQ ID NO 102 | SEQ ID NO 103 | SEQ ID NO 104 | SEQ ID NO 105 |
| PERLD1 | SEQ ID NO 106 | SEQ ID NO 107 | SEQ ID NO 108 | SEQ ID NO 109 | SEQ ID NO 110 |
| PERLD1 | SEQ ID NO 111 | SEQ ID NO 112 | SEQ ID NO 113 | SEQ ID NO 114 | SEQ ID NO 115 |
| AKAP2 | SEQ ID NO 116 | SEQ ID NO 117 | SEQ ID NO 118 | SEQ ID NO 119 | SEQ ID NO 120 |
| AKAP2 | SEQ ID NO 121 | SEQ ID NO 122 | SEQ ID NO 123 | SEQ ID NO 124 | SEQ ID NO 125 |
| AKAP2 | SEQ ID NO 126 | SEQ ID NO 127 | SEQ ID NO 128 | SEQ ID NO 129 | SEQ ID NO 130 |
| AKAP2 | SEQ ID NO 131 | SEQ ID NO 132 | SEQ ID NO 133 | SEQ ID NO 134 | SEQ ID NO 135 |
| ANAPC1 | SEQ ID NO 136 | SEQ ID NO 137 | SEQ ID NO 138 | SEQ ID NO 139 | SEQ ID NO 140 |
| ANAPC1 | SEQ ID NO 141 | SEQ ID NO 142 | SEQ ID NO 143 | SEQ ID NO 144 | SEQ ID NO 145 |
| ANKRD47 | SEQ ID NO 146 | SEQ ID NO 147 | SEQ ID NO 148 | SEQ ID NO 149 | SEQ ID NO 150 |
| ANKRD47 | SEQ ID NO 151 | SEQ ID NO 152 | SEQ ID NO 153 | SEQ ID NO 154 | SEQ ID NO 155 |
| ANKRD47 | SEQ ID NO 156 | SEQ ID NO 157 | SEQ ID NO 158 | SEQ ID NO 159 | SEQ ID NO 160 |
| ATF7 | SEQ ID NO 161 | SEQ ID NO 162 | SEQ ID NO 163 | SEQ ID NO 164 | SEQ ID NO 165 |
| ATF7 | SEQ ID NO 166 | SEQ ID NO 167 | SEQ ID NO 168 | SEQ ID NO 169 | SEQ ID NO 170 |
| ATP5G1 | SEQ ID NO 171 | SEQ ID NO 172 | SEQ ID NO 173 | SEQ ID NO 174 | SEQ ID NO 175 |
| ATP5G1 | SEQ ID NO 176 | SEQ ID NO 177 | SEQ ID NO 178 | SEQ ID NO 179 | SEQ ID NO 180 |
| BCL2 | SEQ ID NO 181 | SEQ ID NO 182 | SEQ ID NO 183 | SEQ ID NO 184 | SEQ ID NO 185 |
| BCL2 | SEQ ID NO 186 | SEQ ID NO 187 | SEQ ID NO 188 | SEQ ID NO 189 | SEQ ID NO 190 |
| BCL2 | SEQ ID NO 191 | SEQ ID NO 192 | SEQ ID NO 193 | SEQ ID NO 194 | SEQ ID NO 195 |
| BCL2 | SEQ ID NO 196 | SEQ ID NO 197 | SEQ ID NO 198 | SEQ ID NO 199 | SEQ ID NO 200 |
| C9orf3 | SEQ ID NO 201 | SEQ ID NO 202 | SEQ ID NO 203 | SEQ ID NO 204 | SEQ ID NO 205 |
| C9orf3 | SEQ ID NO 206 | SEQ ID NO 207 | SEQ ID NO 208 | SEQ ID NO 209 | SEQ ID NO 210 |
| COIL | SEQ ID NO 211 | SEQ ID NO 212 | SEQ ID NO 213 | SEQ ID NO 214 | SEQ ID NO 215 |
| COIL | SEQ ID NO 216 | SEQ ID NO 217 | SEQ ID NO 218 | SEQ ID NO 219 | SEQ ID NO 220 |
| CSF2 | SEQ ID NO 221 | SEQ ID NO 222 | SEQ ID NO 223 | SEQ ID NO 224 | SEQ ID NO 225 |
| CSF2 | SEQ ID NO 226 | SEQ ID NO 227 | SEQ ID NO 228 | SEQ ID NO 229 | SEQ ID NO 230 |
| DMP1 | SEQ ID NO 231 | SEQ ID NO 232 | SEQ ID NO 233 | SEQ ID NO 234 | SEQ ID NO 235 |
| DMP1 | SEQ ID NO 236 | SEQ ID NO 237 | SEQ ID NO 238 | SEQ ID NO 239 | SEQ ID NO 240 |
| E2F3 | SEQ ID NO 241 | SEQ ID NO 242 | SEQ ID NO 243 | SEQ ID NO 244 | SEQ ID NO 245 |
| E2F3 | SEQ ID NO 246 | SEQ ID NO 247 | SEQ ID NO 248 | SEQ ID NO 249 | SEQ ID NO 250 |
| E2F3 | SEQ ID NO 251 | SEQ ID NO 252 | SEQ ID NO 253 | SEQ ID NO 254 | SEQ ID NO 255 |
| ELMO2 | SEQ ID NO 256 | SEQ ID NO 257 | SEQ ID NO 258 | SEQ ID NO 259 | SEQ ID NO 260 |
| ELMO2 | SEQ ID NO 261 | SEQ ID NO 262 | SEQ ID NO 263 | SEQ ID NO 264 | SEQ ID NO 265 |
| ERBB2 | SEQ ID NO 266 | SEQ ID NO 267 | SEQ ID NO 268 | SEQ ID NO 269 | SEQ ID NO 270 |
| ERBB2 | SEQ ID NO 271 | SEQ ID NO 272 | SEQ ID NO 273 | SEQ ID NO 274 | SEQ ID NO 275 |
| ERBB2 | SEQ ID NO 276 | SEQ ID NO 277 | SEQ ID NO 278 | SEQ ID NO 279 | SEQ ID NO 280 |
| ERBB2 | SEQ ID NO 281 | SEQ ID NO 282 | SEQ ID NO 283 | SEQ ID NO 284 | SEQ ID NO 285 |
| ERBB2 | SEQ ID NO 286 | SEQ ID NO 287 | SEQ ID NO 288 | SEQ ID NO 289 | SEQ ID NO 290 |
| GABRG2 | SEQ ID NO 291 | SEQ ID NO 292 | SEQ ID NO 293 | SEQ ID NO 294 | SEQ ID NO 295 |
| GABRG2 | SEQ ID NO 296 | SEQ ID NO 297 | SEQ ID NO 298 | SEQ ID NO 299 | SEQ ID NO 300 |
| GPC5 | SEQ ID NO 301 | SEQ ID NO 302 | SEQ ID NO 303 | SEQ ID NO 304 | SEQ ID NO 305 |
| GPC5 | SEQ ID NO 306 | SEQ ID NO 307 | SEQ ID NO 308 | SEQ ID NO 309 | SEQ ID NO 310 |
| GPC5 | SEQ ID NO 311 | SEQ ID NO 312 | SEQ ID NO 313 | SEQ ID NO 314 | SEQ ID NO 315 |
| GPC5 | SEQ ID NO 316 | SEQ ID NO 317 | SEQ ID NO 318 | SEQ ID NO 319 | SEQ ID NO 320 |
| HSPA6 | SEQ ID NO 321 | SEQ ID NO 322 | SEQ ID NO 323 | SEQ ID NO 324 | SEQ ID NO 325 |
| HSPA6 | SEQ ID NO 326 | SEQ ID NO 327 | SEQ ID NO 328 | SEQ ID NO 329 | SEQ ID NO 330 |
| KIAA0100 | SEQ ID NO 331 | SEQ ID NO 332 | SEQ ID NO 333 | SEQ ID NO 334 | SEQ ID NO 335 |
| KIAA0100 | SEQ ID NO 336 | SEQ ID NO 337 | SEQ ID NO 338 | SEQ ID NO 339 | SEQ ID NO 340 |
| KISS1 | SEQ ID NO 341 | SEQ ID NO 342 | SEQ ID NO 343 | SEQ ID NO 344 | SEQ ID NO 345 |
| KISS1 | SEQ ID NO 346 | SEQ ID NO 347 | SEQ ID NO 348 | SEQ ID NO 349 | SEQ ID NO 350 |
| KISS1 | SEQ ID NO 351 | SEQ ID NO 352 | SEQ ID NO 353 | SEQ ID NO 354 | SEQ ID NO 355 |
| MARCH3 | SEQ ID NO 356 | SEQ ID NO 357 | SEQ ID NO 358 | SEQ ID NO 359 | SEQ ID NO 360 |
| MARCH3 | SEQ ID NO 361 | SEQ ID NO 362 | SEQ ID NO 363 | SEQ ID NO 364 | SEQ ID NO 365 |
| MRO | SEQ ID NO 366 | SEQ ID NO 367 | SEQ ID NO 368 | SEQ ID NO 369 | SEQ ID NO 370 |
| MRO | SEQ ID NO 371 | SEQ ID NO 372 | SEQ ID NO 373 | SEQ ID NO 374 | SEQ ID NO 375 |
| MSX1 | SEQ ID NO 376 | SEQ ID NO 377 | SEQ ID NO 378 | SEQ ID NO 379 | SEQ ID NO 380 |
| MSX1 | SEQ ID NO 381 | SEQ ID NO 382 | SEQ ID NO 383 | SEQ ID NO 384 | SEQ ID NO 385 |
| MSX1 | SEQ ID NO 386 | SEQ ID NO 387 | SEQ ID NO 388 | SEQ ID NO 389 | SEQ ID NO 390 |
| MSX1 | SEQ ID NO 391 | SEQ ID NO 392 | SEQ ID NO 393 | SEQ ID NO 394 | SEQ ID NO 395 |
| MSX1 | SEQ ID NO 396 | SEQ ID NO 397 | SEQ ID NO 398 | SEQ ID NO 399 | SEQ ID NO 400 |
| MSX1 | SEQ ID NO 401 | SEQ ID NO 402 | SEQ ID NO 403 | SEQ ID NO 404 | SEQ ID NO 405 |
| MSX1 | SEQ ID NO 406 | SEQ ID NO 407 | SEQ ID NO 408 | SEQ ID NO 409 | SEQ ID NO 410 |
| MSX1 | SEQ ID NO 411 | SEQ ID NO 412 | SEQ ID NO 413 | SEQ ID NO 414 | SEQ ID NO 415 |
| MSX1 | SEQ ID NO 416 | SEQ ID NO 417 | SEQ ID NO 418 | SEQ ID NO 419 | SEQ ID NO 420 |
| MSX1 | SEQ ID NO 421 | SEQ ID NO 422 | SEQ ID NO 423 | SEQ ID NO 424 | SEQ ID NO 425 |
| MSX1 | SEQ ID NO 426 | SEQ ID NO 427 | SEQ ID NO 428 | SEQ ID NO 429 | SEQ ID NO 430 |
| MSX1 | SEQ ID NO 431 | SEQ ID NO 432 | SEQ ID NO 433 | SEQ ID NO 434 | SEQ ID NO 435 |
| NCOA6 | SEQ ID NO 436 | SEQ ID NO 437 | SEQ ID NO 438 | SEQ ID NO 439 | SEQ ID NO 440 |
| NCOA6 | SEQ ID NO 441 | SEQ ID NO 442 | SEQ ID NO 443 | SEQ ID NO 444 | SEQ ID NO 445 |
| PAPOLA | SEQ ID NO 446 | SEQ ID NO 447 | SEQ ID NO 448 | SEQ ID NO 449 | SEQ ID NO 450 |
| PAPOLA | SEQ ID NO 451 | SEQ ID NO 452 | SEQ ID NO 453 | SEQ ID NO 454 | SEQ ID NO 455 |
| PDGFRA | SEQ ID NO 456 | SEQ ID NO 457 | SEQ ID NO 458 | SEQ ID NO 459 | SEQ ID NO 460 |
| PDGFRA | SEQ ID NO 461 | SEQ ID NO 462 | SEQ ID NO 463 | SEQ ID NO 464 | SEQ ID NO 465 |
| PDGFRA | SEQ ID NO 466 | SEQ ID NO 467 | SEQ ID NO 468 | SEQ ID NO 469 | SEQ ID NO 470 |
| RPL23A | SEQ ID NO 471 | SEQ ID NO 472 | SEQ ID NO 473 | SEQ ID NO 474 | SEQ ID NO 475 |
| RPL23A | SEQ ID NO 476 | SEQ ID NO 477 | SEQ ID NO 478 | SEQ ID NO 479 | SEQ ID NO 480 |
| RPL23A | SEQ ID NO 481 | SEQ ID NO 482 | SEQ ID NO 483 | SEQ ID NO 484 | SEQ ID NO 485 |
| RPL23A | SEQ ID NO 486 | SEQ ID NO 487 | SEQ ID NO 488 | SEQ ID NO 489 | SEQ ID NO 490 |
| RUFY3 | SEQ ID NO 491 | SEQ ID NO 492 | SEQ ID NO 493 | SEQ ID NO 494 | SEQ ID NO 495 |
| RUFY3 | SEQ ID NO 496 | SEQ ID NO 497 | SEQ ID NO 498 | SEQ ID NO 499 | SEQ ID NO 500 |
| SOX3 | SEQ ID NO 501 | SEQ ID NO 502 | SEQ ID NO 503 | SEQ ID NO 504 | SEQ ID NO 505 |
| SOX3 | SEQ ID NO 506 | SEQ ID NO 507 | SEQ ID NO 508 | SEQ ID NO 509 | SEQ ID NO 510 |
| TP53 | SEQ ID NO 511 | SEQ ID NO 512 | SEQ ID NO 513 | SEQ ID NO 514 | SEQ ID NO 515 |
| TP53 | SEQ ID NO 516 | SEQ ID NO 517 | SEQ ID NO 518 | SEQ ID NO 519 | SEQ ID NO 520 |
| TSPYL1 | SEQ ID NO 521 | SEQ ID NO 522 | SEQ ID NO 523 | SEQ ID NO 524 | SEQ ID NO 525 |
| TSPYL1 | SEQ ID NO 526 | SEQ ID NO 527 | SEQ ID NO 528 | SEQ ID NO 529 | SEQ ID NO 530 |
| UBXD3 | SEQ ID NO 531 | SEQ ID NO 532 | SEQ ID NO 533 | SEQ ID NO 534 | SEQ ID NO 535 |
| UBXD3 | SEQ ID NO 536 | SEQ ID NO 537 | SEQ ID NO 538 | SEQ ID NO 539 | SEQ ID NO 540 |
| ZNF420 | SEQ ID NO 541 | SEQ ID NO 542 | SEQ ID NO 543 | SEQ ID NO 544 | SEQ ID NO 545 |
| ZNF420 | SEQ ID NO 546 | SEQ ID NO 547 | SEQ ID NO 548 | SEQ ID NO 549 | SEQ ID NO 550 |

### FIGURES

FIG. 1: Disease free survival depending on cg19176447 methylation.

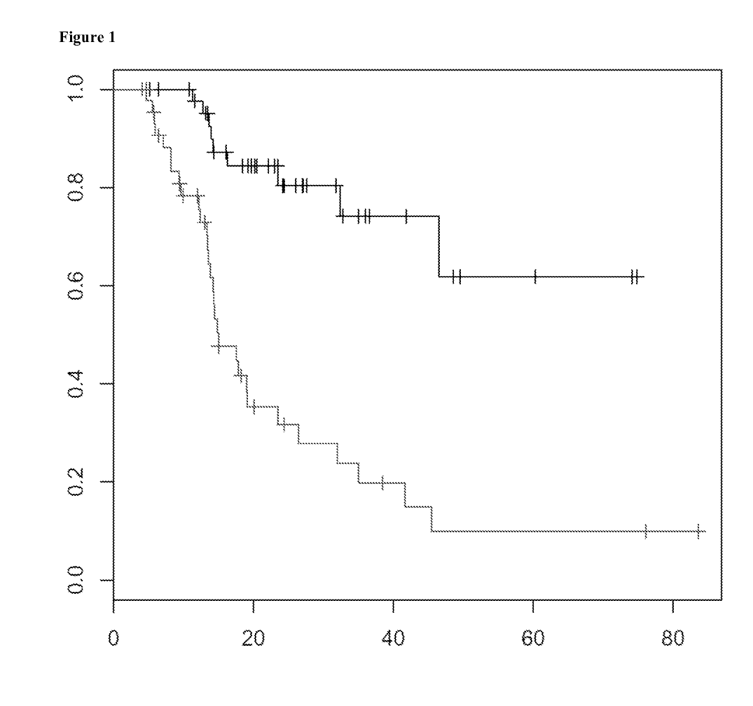

FIG. 2: Disease free survival depending on cg23906291 methylation.

FIG. 3: Disease free survival depending on cg06740897 methylation.

FIG. 4: Disease free survival depending on cg07318658 methylation.

FIG. 5: Disease free survival depending on cg19510604 methylation.

FIG. 6: Disease free survival depending on combined methylation.

### EXAMPLES

### Patient Samples

### Cancer Patient/Cancer Samples/Clinical Data

Ovarian cancer samples were obtained from patients treated at the Department of Obstetrics and Gynaecology, Charite University Hospital, Campus Virchow, Berlin. Tumor tissue was snap frozen to −80° C. Patients gave their written informed consent and the study was approved by the local institutional review boards.

Patients with primary ovarian cancer of serous subtype (n=90) were selected who after debulking surgery received standard 1^{st} line therapy with carboplatin and paclitaxel or cisplatin and paclitaxel. All primary ovarian tumor specimens were inspected by independent pathologists who confirmed the original diagnosis, assessed the sample quality, and assessed the amount of tumor tissue, stromal tissue and normal tissue. Clinical data including base data, treatment data and follow-up data were available from all patients that were used for methylation analysis.

Other cancer samples and benign control samples and pathologically verified tissues were obtained from commercial sample collections done by Asterand (Detroit, Mich., USA).

### Sample Processing

DNA extraction was performed using QIAamp DNA Mini Kit, QIAGEN GmbH (Hilden, Germany) according to the manufacturer's instructions. Total genomic DNA of all samples was bisulfite treated, converting unmethylated cytosines to uracil. Methylated cytosines remained conserved. Bisulfite treatment was performed using the EZ-96 DNA Methylation-Gold Kit from Zymo Research (Orange, Calif. USA) according to the manufacturer's instructions. The bisulfite treated DNA was whole genome amplified via random hexamer priming and Phi29 DNA polymerase, enzymatically fragmented and hybridized to Illumina Infinium II BeadChips. Target preparation and microarray hybridization, washing, and scanning was performed according to the manufacturer's protocol (Illumina, Inc., San Diego, Calif., USA). The methylation values for each CpG dinucleotide were calculated from the raw data using the BeadStudio software (Illumina, Inc., San Diego, Calif., USA).

### Bioinformatics Methods

All calculations were made with the statistics software “R” in combination with its “Bioconductor” packages (http://www.bioconductor.org/about/index.html). R is a standard software for statistical computations from the open source project “The R Project for Statistical Computing” (http://cran.r-project.org/). The version of R used was 2.7.2, that of Bioconductor was 2.2.

All Kaplan-Meier estimates (Kaplan, E. L.; Meier, P.: Nonparametric estimation from incomplete observations. J. Amer. Statist. Assn. 53:457-481, 1958) of survival functions were produced by Bioconductor's package “survival” with the function “survfit”. Argument data were the disease-free survival data and the methylation measurements of a specific marker. The group of highly methylated samples for that marker (“MethHigh”) always consists of the 50% of samples with measurements above the median of all measurements for that marker.

For combined sequences, a weighted average of ranks of the measurements of the combined sequences is used as a combined measurement. The upper 50% of the combined measurements are seen as highly methylated. This is adapted as follows when combining sequences with reverse predictive qualities for survival, such as a sequence for which high methylation means longer survival and a sequence for which low methylation means longer survival. Then one of the sequence's measurements is ranked in reverse order before the forming of the average ranks These methods of combining two sequences are analogously extended to the combination of greater number of sequences. For the weighted average only equal weights were used in the example in this patent.

The logrank test (Mantel, Nathan (1966). “Evaluation of survival data and two new rank order statistics arising in its consideration.” Cancer Chemotherapy Reports 50 (3): 163-70. PMID 5910392) for differences between survival functions (that is, with a null hypothesis of equality between the survival functions) was computed by the function “survdiff” from the same package with the same arguments as “survfit” above.

Finally, the fit of the Cox proportional hazard model for multiple categorial factors was computed by the function “coxph” from the same package. Arguments were the disease free survival data for the available tumor samples, the median-based factor “MethHigh” explained above, the factorization of FIGO stage data into the class of cases with stage I or stage II and the class with stage III or IV (the latter being called “StageHigh”), and the factorization of tumor rest after surgery into the class of cases with no tumor rest and the class with some tumor rest (called “TumorRest”).

### Results

1. Determining the Prognosis of a Subject with Ovarian Carcinoma Based on Methylation of the Ggene CLK3

The prognosis of a subject with ovarian carcinoma was determined using SEQ ID NO 1, which is part of the gene CLK3.

SEQ ID No 1 is present on the HumanMethylation27 BeadChip (Illumina© Infinium, http://www.illumina.com/products/infinium_humanmethylation27_beadchip_kits.ilmn) and identified as “cg19510604”. This CpG is present in the genomic sequences as SEQ ID NO 1, and as part of SEQ ID NO 6, and in close proximity to SEQ ID NO 11. A high correlation of methylation in all three sites is expected.

Fehler! Verweisquelle Konnte Nicht Gefunden Werden.

shows two Kaplan-Meier curves for disease free survival in months, the black curve corresponds to samples with a low methylation of cg19510604, the lighter curve corresponds to those with a high methylation. The cut-off point between high and low methylation was chosen to be the median of the 90 available tumor samples, with 45 samples in the low group (with 30 events) and 45 in the high group (with 9 events).

A logrank test for differences between the two survival curves results in a chi-square statistic for a test of equality of 24.4 on 1 degrees of freedom, p=7.89*10⁻⁷.

A Cox proportional hazard regression model for the three factors “high methylation of cg19510604”, “FIGO Stage III or IV”, “Tumor rest remaining after surgery” results in the following (Likelihood ratio test resulted in 29.4 on three degrees of freedom, p-Value of 1.83*10⁻⁶.):

| | Coef- | exp(Coef- | SE(Coef- | | |
|---|---|---|---|---|---|
| | ficient | ficient) | ficient) | Z-Score | p-Value |
| high | 1.529 | 4.61 | 0.404 | 3.782 | 0.00016 |
| Methylation | | | | | |
| Stage III or IV | 0.358 | 1.43 | 1.061 | 0.337 | 0.74000 |
| Tumor Rest | 0.807 | 2.24 | 0.341 | 2.363 | 0.01800 |

2. Determining the Prognosis of a Subject with Ovarian Carcinoma Based on the Methylation of the Gene MTMR4

The prognosis of a subject with ovarian carcinoma was determined using SEQ ID NO 16, which is part of the gene MTMR4.

SEQ ID NO 16 is present on the HumanMethylation27 BeadChip (Illumina© Infinium, http://www.illumina.com/products/infinium_humanmethylation27_beadchip_kits.ilmn) and identified as “cg07318658”. This CpG is present in the genomic sequences as SEQ ID NO 16, and as part of SEQ ID NO 21, and in close proximity to SEQ ID NOs 26, 31, and 36. A high correlation of methylation in all five sites is expected.

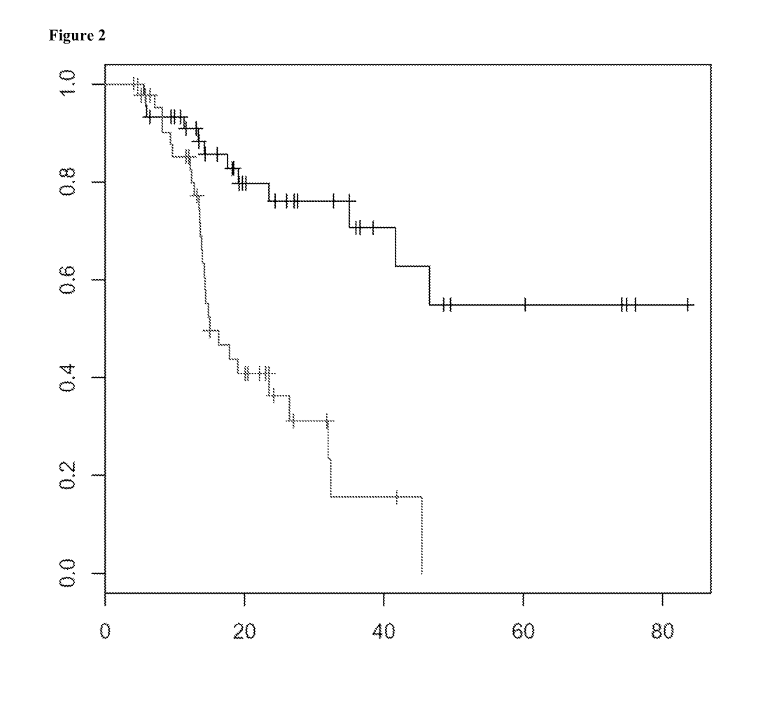

FIG. 2 shows two Kaplan-Meier curves for disease free survival in months, the black curve corresponds to samples with a low methylation of cg07318658, the lighter curve corresponds to those with a high methylation. The cut-off point between high and low methylation was chosen to be the median of the available tumor samples, with 45 samples in the low group (with 27 events) and 45 in the high group (with 12 events).

A logrank test for differences between the two survival curves results in a chi-square statistic for a test of equality of 18.0 on 1 degrees of freedom, p=2.2*10⁻⁵.

A Cox proportional hazard regression model for the three factors “high methylation of cg07318658”, “FIGO Stage III or IV”, “Tumor rest remaining after surgery” results in the following (Likelihood ratio test resulted in 31.0 on three degrees of freedom, p-Value of 8.67*10⁻⁶):

| | Coef- | exp(Coef- | SE(Coef- | | |
|---|---|---|---|---|---|
| | ficient | ficient) | ficient) | Z-Score | p-Value |
| high | 1.550 | 4.720 | 0.379 | 4.100 | 0.000041 |
| Methylation | | | | | |
| Stage III or IV | 1.130 | 3.110 | 1.018 | 1.110 | 0.27 |
| Tumor Rest | 1.170 | 3.220 | 0.344 | 3.400 | 0.00069 |

3. Determining the Prognosis of a Subject with Ovarian Carcinoma Based on Methylation of the Gene NFE2L1

The prognosis of a subject with ovarian carcinoma was determined using SEQ ID NO 41, which is part of gene NFE2L1.

SEQ ID NO 41 is present on the HumanMethylation27 BeadChip (Illumina© Infinium, http://www.illumina.com/products/infinium_humanmethylation27_beadchip_kits.ilmn) and identified as “cg06740897”. This CpG is present in the genomic sequences as SEQ ID NO 41, and as part of SEQ ID NO 46, and in close proximity to SEQ ID NOs 51, 56, and 61. A high correlation of methylation in all five sites is expected.

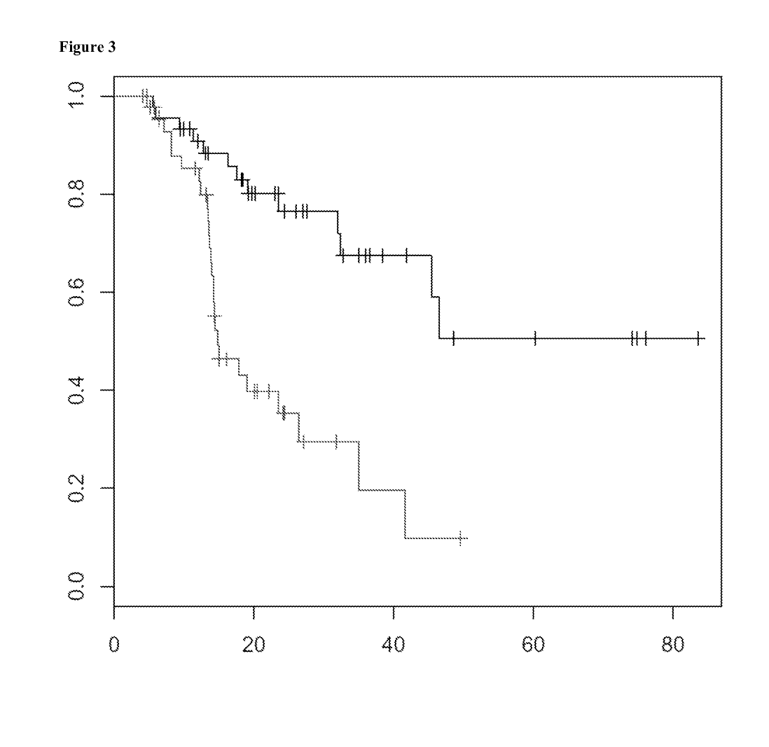

FIG. 3 shows two Kaplan-Meier curves for disease free survival in months, the black curve corresponds to samples with a low methylation of cg06740897, the lighter curve corresponds to those with a high methylation. The cut-off point between high and low methylation was chosen to be the median of the 90 available tumor samples, with 45 samples in the low group (with 13 events) and 45 in the high group (with 26 events).

A logrank test for differences between the two survival curves results in a chi-square statistic for a test of equality of 16.2 on 1 degrees of freedom, p=5.8*10⁻⁵.

A Cox proportional hazard regression model for the three factors “high methylation of cg 06740897”, “FIGO Stage III or IV”, “Tumor rest remaining after surgery” results in the following (Likelihood ratio test resulted in 24.8 on three degrees of freedom, p-Value of 1.74*10⁻⁵):

| | Coef- | exp(Coef- | SE(Coef- | | |
|---|---|---|---|---|---|
| | ficient | ficient) | ficient) | Z-Score | p-Value |
| high | 1.231 | 3.430 | 0.355 | 3.470 | 0.00052 |
| Methylation | | | | | |
| Stage III or IV | 1.386 | 4.000 | 1.015 | 1.370 | 0.17 |
| Tumor Rest | 0.897 | 2.450 | 0.339 | 2.640 | 0.0083 |

4. Determining the Prognosis of a Subject with Ovarian Carcinoma Based on Methylation of the Gene PERLD1

The prognosis of a subject with ovarian carcinoma was determined using SEQ ID NO 66, which is part of gene PERLD1.

SEQ ID NO 66 is present on the HumanMethylation27 BeadChip (Illumina© Infinium, http://www.illumina.com/products/infinium_humanmethylation27_beadchip_kits.ilmn) and identified as “cg23906291”. This CpG is present in the genomic sequences as SEQ ID NO 66, and as part of SEQ ID NOs 71, 76, 101, and 111, and in close proximity of SEQ ID NOs 81, 86, 91, 96, and 106. A high correlation of methylation in all five sites is expected.

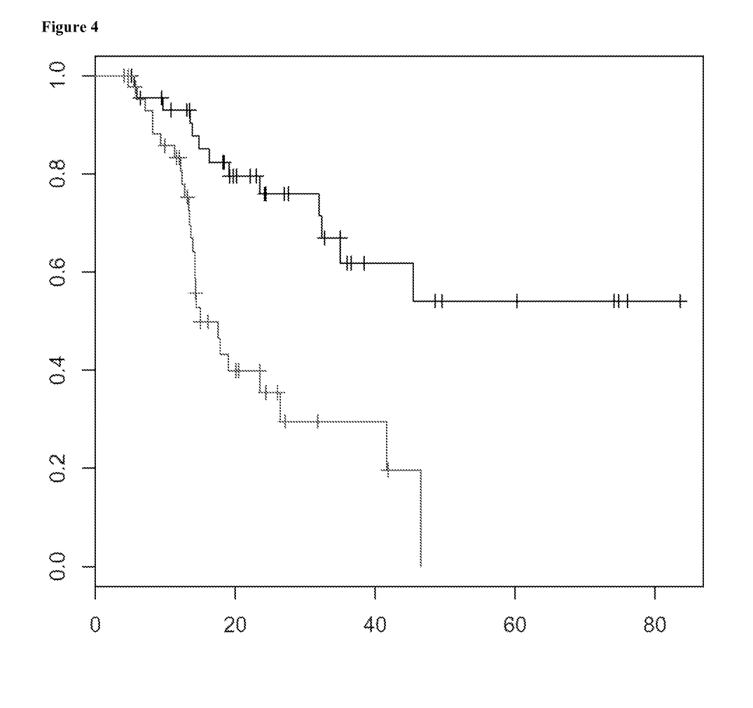

FIG. 4 shows two Kaplan-Meier curves for disease free survival in months, the black curve corresponds to samples with a low methylation of cg23906291, the red curve corresponds to those with a high methylation. The cut-off point between high and low was chosen to be the median of the available 90 tumor samples, with 45 samples in the low group (with 13 events) and 45 in the high group (with 26 events).

A logrank test for differences between the two survival curves results in a chi-square statistic for a test of equality of 15.7 on 1 degrees of freedom, p=7.54*10⁻⁵.

A Cox proportional hazard regression model for the three factors “high methylation of cg23906291”, “FIGO Stage III or IV”, “Tumor rest remaining after surgery” results in the following (Likelihood ratio test resulted in 29.5 on three degrees of freedom, p-Value of 1.80*10⁻⁶):

| | Coef- | exp(Coef- | SE(Coef- | | |
|---|---|---|---|---|---|
| | ficient | ficient) | ficient) | Z-Score | p-Value |
| high | 1.460 | 4.300 | 0.362 | 4.030 | 0.00005.6 |
| Methylation | | | | | |
| Stage III or IV | 1.520 | 4.590 | 1.021 | 1.490 | 0.14 |
| Tumor Rest | 1.250 | 3.490 | 0.350 | 3.570 | 0.00035 |

5. Determining the Prognosis of a Subject with Ovarian Carcinoma Based on Methylation of the Gene AKAP2

The prognosis of a subject with ovarian carcinoma was determined using SEQ ID NO 116, which is part of gene AKAP2.

SEQ ID NO 116 is present on the HumanMethylation27 BeadChip (Illumina© Infinium, http://www.illumina.com/products/infinium_humanmethylation27_beadchip_kits.ilmn) and identified as “cg19176447”. This CpG is present in the genomic sequences as SEQ ID NO 116, as part of SEQ ID NO 121, and in close proximity of SEQ ID NOs 126 and 131. A high correlation of methylation in all five sites is expected.

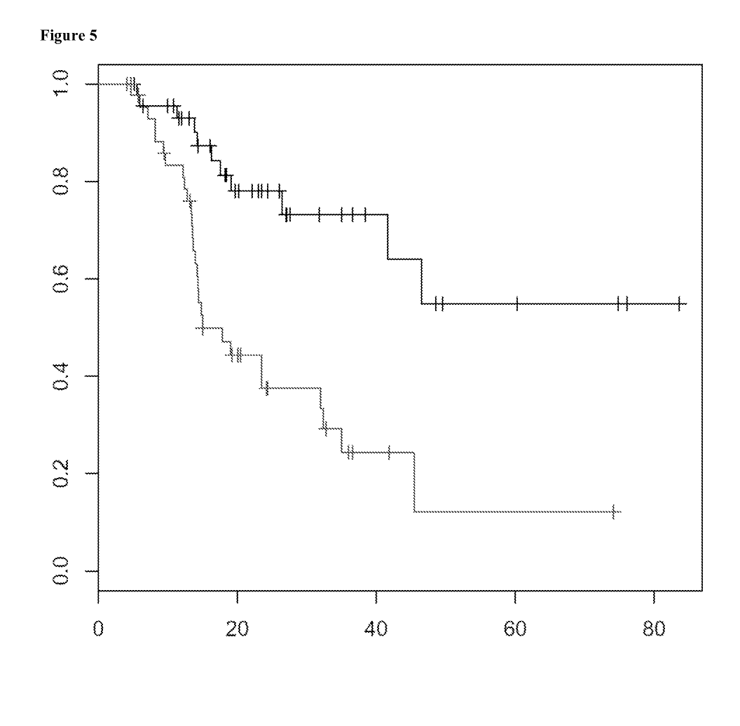

FIG. 5 shows two Kaplan-Meier curves for disease free survival in months, the black (upper) curve corresponds to samples with a low methylation of cg19176447, the lower (lighter) curve corresponds to those with a high methylation. The cut-off point between high and low was chosen to be the median of the available 90 tumor samples, with 45 samples in the low group (with 11 events) and 45 in the high group (with 28 events).

A logrank test for differences between the two survival curves results in a chi-square statistic for a test of equality of 14.1 on 1 degrees of freedom, p=1.77*10⁻⁴.

A Cox proportional hazard regression model for the three factors “high methylation of cg19176447”, “FIGO Stage III or IV”, “Tumor rest remaining after surgery” results in the following (Likelihood ratio test resulted in 17.4 on three degrees of freedom, p-Value of 5.74*10⁻⁴):

| | Coef- | exp(Coef- | SE(Coef- | | |
|---|---|---|---|---|---|
| | ficient | ficient) | ficient) | Z-Score | p-Value |
| high Methyl. | 1.190 | 3.290 | 0.366 | 3.250 | 0.0011 |
| Stage III or IV | 1.447 | 4.250 | 1.015 | 1.430 | 0.15 |
| Tumor Rest | 0.872 | 2.390 | 0.342 | 2.550 | 0.011 |

6. Determining the Prognosis of a Subject with Ovarian Carcinoma Based on Methylation of Gene CLK3 and Gene MTMR4

The prognosis of a subject with ovarian carcinoma was determined using SEQ ID NO 1, which is part of gene CLK3 in combination with SEQ ID NO 16, which is part of gene MTMR4. Each of the sequences has further sequences in Table 1 that are expected to be closely correlated.

Specifically, genomic SEQ ID NO 1 and 16 were combined by ranking each sequence's methylation measurements from the HumanMethylation27 BeadChip (All measurements were performed using the Illumina© Infinium HumanMethylation27 BeadChip:http://www.illumina.com/products/infinium_{—}humanmethylation27_beadchip_kits.ilmn) and then averaging these ranks for each sample.

For the resulting average ranks the median is used as a divider between “MethLow” and “MethHigh” as above.

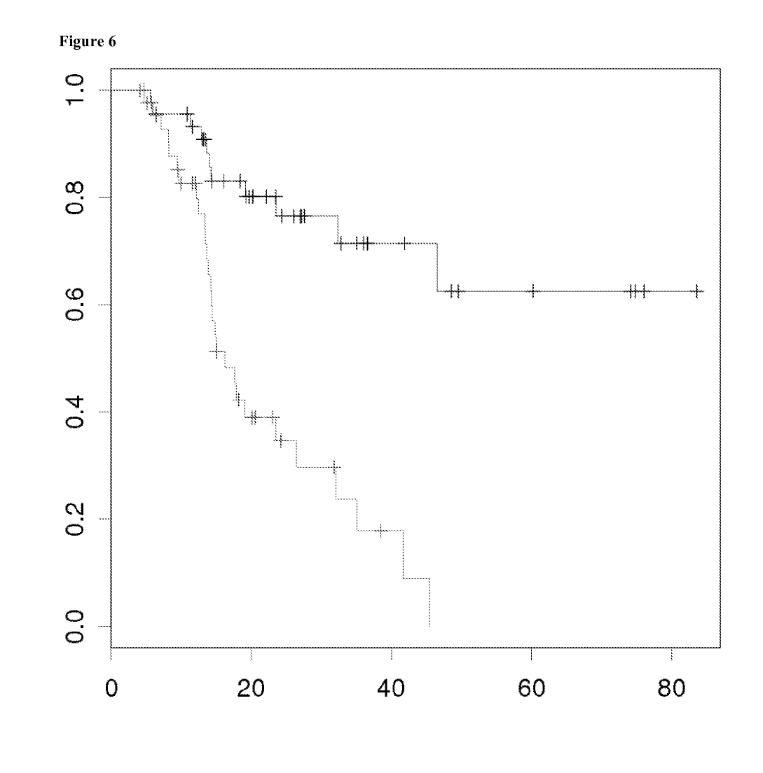

FIG. 6 shows two Kaplan-Meier curves for disease free survival in months, the black curve corresponds to samples with a low combined methylation, the lighter curve corresponds to those with a high methylation. The cut-off point between high and low methylation was chosen to be the median of the 90 available tumor samples, with 45 samples in the low group (with 11 events) and 45 in the high group (with 28 events).

A logrank test for differences between the two survival curves results in a chi-square statistic for a test of equality of 21.9 on 1 degrees of freedom, p=2.91*10-6.

A Cox proportional hazard regression model for the three factors “high average rank of methylation”, “FIGO Stage III or IV”, “Tumor rest remaining after surgery” results in the following (likelihood ratio test resulted in 17.4 on three degrees of freedom, p-Value of 5.74*10-4.):

| | Coef- | exp(Coef- | SE(Coef- | | |
|---|---|---|---|---|---|
| | ficient | ficient) | ficient) | Z-Score | p-Value |
| high | 1.395 | 4.03 | 0.39 | 3.758 | 0.00035 |
| average Rank | | | | | |
| Stage III or IV | 0.821 | 2.27 | 1.025 | 0.801 | 0.42 |
| Tumor Rest | 0.707 | 2.03 | 0.345 | 2.047 | 0.041 |

1. A method for determining the prognosis of a subject with ovarian carcinoma using a biological sample comprising genomic tumor DNA isolated from the subject, comprising:
determining the methylation status of a CpG dinucleotide in a target sequence that is selected from the group consisting of the target sequences as referred to by gene name in Table 1 in a biological sample isolated from a subject, and
deducing from the determined methylation status of the target sequence the prognosis of the subject with ovarian carcinoma.

2. The method of claim 1, wherein the subject with ovarian carcinoma receives a platinum-based treatment.

3. The method according to claim 1, wherein the methylation status of at least 2, 5, 10, 15, or 25 CpG dinucleotides are determined.

4. The method according to claim 1, wherein the biological sample is selected from the group consisting of ovarian tumor tissue, lymph node metastasis tissue, blood, serum, plasma, peritoneal cavity fluid.

5. The method according to claim 1, further comprising isolating genomic DNA from the biological sample.

6. The method according to claim 1, wherein determining the methylation status comprises treating the genomic DNA or a fragment thereof with a chemical reagent or an enzyme containing solution, whereby the base pairing behavior of methylated cytosine bases and/or unmethylated cytosine bases of the nucleic acid are altered such that methylated cytosine bases become distinguishable from unmethylated cytosine bases.

7. The method according to claim 1, wherein determining the methylation status comprises amplifying the treated genomic DNA by means of methylation specific primers and/or blocking oligonucleotides.

8. The method according to claim 7, wherein determining the methylation status comprises determining the presence or absence of the amplified DNA by means of a real-time detection probe.

9. The method according to claim 1, wherein determining the methylation status comprises determining the methylation status of a CpG dinucleotide in at least two target sequences, in a biological sample isolated from a subject.

10. A nucleic acid for the detection of ovarian carcinoma, wherein the nucleic acid comprises at least 16 contiguous nucleotides of a sequence selected from the group consisting of the bisulfite sequences of Table 1, and sequences complementary thereto.

11. A nucleic acid for detecting ovarian cancer in a subject, comprising at least 50 contiguous nucleotides of a sequence selected from the group consisting of the bisulfite sequences as provided in Table 1, and sequences complementary thereto.

12. A kit for determining the prognosis of subjects with ovarian carcinoma comprising
(a) a bisulfite reagent for converting a nucleic acid, and
(b) a set of oligonucleotides comprising two oligonucleotides wherein the sequence of each of the two oligonucleotides is identical, is complementary, or hybridizes under stringent or highly stringent conditions to an at least 9 base long segment of a sequence selected from the bisulfite sequences of Table 1.

13. A kit for determining the prognosis of subjects with ovarian carcinoma comprising
(a) a methylation sensitive restriction enzyme reagent, and
(b) at least one set of oligonucleotides comprising one or a plurality of nucleic acids or peptide nucleic acids which are identical, are complementary, or hybridize under stringent or highly stringent conditions to an at least 9 base long segment of the genomic sequences as provided in Table 1.

14. (canceled)

15. The method of claim 9 wherein the two target sequences are selected from the group consisting of the target sequences as referred to by gene name in Table 1.

16. The method of claim 15 wherein the two target sequences are from different genes.

17. The method of claim 12 wherein the sequence of each of the two oligonucleotides is identical, is complementary, or hybridizes under stringent or highly stringent conditions to an at least 18 base long segment of the sequence selected from the bisulfite sequences of Table 1.

18. The method of claim 13 wherein the one or at least one of the plurality of nucleic acids or peptide nucleic acids is identical, is complementary, or hybridizes under stringent or highly stringent conditions to an at least 18 base long segment of the genomic sequences as provided in Table 1.
